SEQUENCE LISTING (18 sequences)

SEQ ID NO: 1 - DNA, 48 bp, synthetic construct
  source 1..48: mol_type = other DNA
  modified_base 1: mod_base = OTHER; note = BiomersNet-Quencher-535
  modified_base 24: mod_base = OTHER; note = Fluorescein amidites
  modified_base 48: mod_base = OTHER; note = Universal Reporter (UR)-Block
attgcgggagatgagacccgcaattgttggtcgtagagcccagaacga

SEQ ID NO: 2 - DNA, 26 bp, synthetic construct
  source 1..26: mol_type = other DNA
  modified_base 1: mod_base = OTHER; note = BiomersNet-Quencher-535
  modified_base 24: mod_base = OTHER; note = BiomersNet-Quencher-535
  modified_base 25: mod_base = OTHER; note = BiomersNet-Quencher-535
gcgggtctcatctcacgctgatgtct

SEQ ID NO: 3 - DNA, 52 bp, synthetic construct
  source 1..52: mol_type = other DNA
agacatcagcgtgagatgagacccgcaatgttggtcgtagagcccagaacga

SEQ ID NO: 4 - DNA, 67 bp, synthetic construct
  source 1..67: mol_type = other DNA
gacagtcgtcgcatgctgcagtgggagagcagaagtccgtgtgcattggtcgtagagcccagaacga

SEQ ID NO: 5 - DNA, 22 bp, synthetic construct
  source 1..22: mol_type = other DNA
  modified_base 22: mod_base = OTHER; note = Fluorescein amidites
tgcacacggacttctgctctcc

SEQ ID NO: 6 - DNA, 23 bp, synthetic construct
  source 1..23: mol_type = other DNA
  modified_base 1: mod_base = OTHER; note = Fluorescein amidites
cactgcagcatgcgacgactgtc

SEQ ID NO: 7 - DNA, 67 bp, synthetic construct; identical to SEQ ID NO: 4
  source 1..67: mol_type = other DNA
  modified_base 24: mod_base = OTHER; note = BiomersNet-Quencher-535

SEQ ID NO: 8 - DNA, 20 bp, synthetic construct
  source 1..20: mol_type = other DNA
gtcgtcagctcgtgttgtga

SEQ ID NO: 9 - DNA, 20 bp, synthetic construct
  source 1..20: mol_type = other DNA
tccccaccttcctccagttt

SEQ ID NO: 10 - DNA, 40 bp, synthetic construct
  source 1..40: mol_type = other DNA
gggctctacgaccaaatgttgggttaagtcccgcaacgag

SEQ ID NO: 11 - DNA, 27 bp, synthetic construct
  source 1..27: mol_type = other DNA
  modified_base 27: mod_base = OTHER; note = Cyanine 5
gccgcatctaatgaggtcgaggagtca

SEQ ID NO: 12 - DNA, 58 bp, synthetic construct
  source 1..58: mol_type = other DNA
  modified_base 1: mod_base = OTHER; note = Black Hole Quencher 1
agtgactcctcgacctcattagatgcggcattcgatcacacaacatgagcatgtgtac

SEQ ID NO: 13 - DNA, 26 bp, synthetic construct
  source 1..26: mol_type = other DNA
  modified_base 26: mod_base = OTHER; note = Fluorescent Dye Oligonucleotide Labeling (ATTO-TEC GmbH)
ccgcgcgtcttggagcagtccttgtt

SEQ ID NO: 14 - DNA, 53 bp, synthetic construct
  source 1..53: mol_type = other DNA
  modified_base 1: mod_base = OTHER; note = BiomersNet-Quencher-1
aacaaggactgctccaagacgcgcggattagcatgtgaggaacacgatgacac

SEQ ID NO: 15 - DNA, 77 bp, synthetic construct
  source 1..77: mol_type = other DNA
  modified_base 29: mod_base = OTHER; note = BiomersNet-Quencher-535 + N-Hydoxy-Succiimid-Ester
  modified_base 1: mod_base = OTHER; note = BiomersNet-Quencher-535
aacaaggactgctccaagacgcgcggagtgactcctcgacctcattagatgcggcagcacctgggacatcgactatt

SEQ ID NO: 16 - DNA, 23 bp, synthetic construct
  source 1..23: mol_type = other DNA
cacatgctcatgttgtgtgatcg

SEQ ID NO: 17 - DNA, 23 bp, synthetic construct
  source 1..23: mol_type = other DNA
gtcatcgtgttcctcacatgcta

SEQ ID NO: 18 - DNA, 20 bp, synthetic construct
  source 1..20: mol_type = other DNA
atagtcgatgtcccaggtgc

(19) United States
(12) Patent Application Publication
(10) Pub. No.: US 2025/0340928 A1
(43) Pub. Date: Nov. 6, 2025

(54) NUCLEIC ACID DETECTION IN A PCR BY MEANS OF A TARGET-SEQUENCE-UNSPECIFIC MODULAR REPORTER COMPLEX

(71) Applicants: ALBERT-LUDWIGS-UNIVERSITÄT FREIBURG, Freiburg (DE); HAHN-SCHICKARD-GESELLSCHAFT FÜR ANGEWANDTE FORSCHUNG E. V., Stuttgart (DE)

(72) Inventors: Felix VON STETTEN, Stuttgart (DE); Tamara PFAFF, Freiburg (DE); Michael LEHNERT, Stuttgart (DE); Martin TROTTER, Stuttgart (DE); Nadine BORST, Stuttgart (DE); Lisa BECHERER, Freiburg (DE); Helena GMOSER, Freiburg (DE)

(73) Assignees: ALBERT-LUDWIGS-UNIVERSITÄT FREIBURG, Freiburg (DE); HAHN-SCHICKARD-GESELLSCHAFT FÜR ANGEWANDTE FORSCHUNG E. V., Stuttgart (DE)

(21) Appl. No.: 18/837,317

(22) PCT Filed: Apr. 21, 2023
(86) PCT No.: PCT/EP2023/060517

§ 371 Date: Aug. 9, 2024

(30) Foreign Application Priority Data
Apr. 22, 2022 (EP) 22169463.1
Aug. 19, 2022 (EP) 22191150.6

(51) Int. Cl. C12Q 1/6818; C12Q 1/6851

(52) U.S. Cl. CPC C12Q 1/6818; C12Q 1/6851

(57) ABSTRACT

The invention relates to a method for detecting at least one target nucleic acid sequence by means of a mediator probe and at least one target sequence-unspecific modular reporter complex, wherein the released mediator sequence binds to a mediator binding site of the target sequence-unspecific modular reporter complex and is extended. A signal change is initiated and detected. The invention also relates to a kit for carrying out this method.

### CROSS-REFERENCE TO RELATED APPLICATIONS

This is the U.S. national stage of international application no. PCT/EP2023/060517, filed Apr. 21, 2023 designating the United States and claiming priority to European patent application nos. EP 22169463.1, filed Apr. 22, 2022 and EP 22191150.6, filed August 19,2022, which are incorporated herein by reference in their entireties.

### INCORPORATION OF SEQUENCE LISTING

This US national stage application contains a Sequence Listing which has been submitted electronically in .XML format and is hereby incorporated by reference in its entirety. Said .XML copy, created on Jan. 21, 2025, is named “7014-2200.xml” and is 25,550 bytes in size. The sequence listing contained in this .XML file is part of the specification and is hereby incorporated by reference herein in its entirety.

### TECHNICAL BACKGROUND

In quantitative detection reactions of PCR products such as real-time PCR (qPCR) or digital PCR (dPCR), target sequences are detected either by dyes that intercalate into the DNA, but bind unspecifically to all DNA double strands present, or by DNA probes that only bind a specific DNA target sequence. These DNA probes generate an optical signal change directly (e.g. TaqMan probes) or indirectly (mediator probes in combination with universal reporter molecules) through their cleavage. Optical detectors detect the light emissions generated during the reaction outside the reaction vessel. These detection systems usually use light-absorbing and light-emitting fluorescent molecules. After excitation by light energy of a certain wavelength, these molecules emit energy in the form of higher wavelengths, which can be detected by detectors. A distinction is made between molecules whose light energy is to be detected in a specific wavelength range (fluorescence donor or fluorophore) and molecules that lead to a decrease in the fluorescence intensity of a fluorophore in close proximity (fluorescence acceptor or quencher). If the spatial proximity between fluorophore and quencher changes, the fluorescence signal changes accordingly, whereby a smaller distance always results in higher quenching (FRET quenching or contact quenching).

The use of target sequence-specific DNA probes in combination with different fluorescent labels also enables the sensitive detection of a plurality of DNA sequences in one reaction (multiplex detection). This usually involves the use of a plurality of DNA probes or detection molecules that carry different fluorophore-quencher combinations, each of which emits a fluorescence signal in a specific wavelength range in the presence of a specific DNA sequence. Further developments of this multiplexing detection also enable the detection of multiple target sequences within a wavelength range by quantitatively observing the fluorescence values or combining them with other parameters (e.g. the readout temperature).

Two well-known one-piece target sequence-specific DNA probes for optical detection are Taqman probes and molecular beacons (Tan et al. 2004; Li et al. 2008; Holland et al. 1991; Rodríguez et al. 2005). These bind in a target sequence-specific manner and are cleaved during PCR, generating a signal. The disadvantage of these probes is their dependence on the target gene sequence, which means that only specific regions of a DNA target sequence, which must meet certain requirements, can be used. For example, attention must be paid to probe length, melting temperature, binding enthalpy, GC content, guanine quenching and complementary sequence fragments. However, these probes are easy to synthesize, as usually only two terminal labels are required. In addition to these target sequence-specific DNA probes, there are also fluorogenic detection molecules that are target sequence-unspecific (e.g. mediator probes and universal reporters). These have the disadvantage that their synthesis is considerably more challenging, but they enable completely new types of sequence detection and have a highly optimized fluorescence signal generation. In addition to these types of one-part detection molecules, there are also two-part, target sequence-specific detection probe systems (light cycler probes). Here, a fluorescence signal between two neighboring DNA probes is shifted into the longer wavelength spectral range via their fluorophores using FRET, provided that both bind correctly to the DNA target sequence and thus create the required distance to each other. Such systems also have the problem of sequence specificity and require extreme fine-tuning in their design.

Molecular beacons consist of an oligonucleotide (oligo) with five to seven complementary bases at both ends and a terminal fluorophore or quencher (Tyagi and Kramer 1996). When the ends are attached by forming a loop due to the complementary bases, the fluorescence molecules are brought into spatial proximity. As a result, the fluorescence is transferred from the donor to the acceptor, whereby the emitted light wavelength is changed or quenched by energy loss into the longer wavelength of the acceptor. Only after binding of the oligo to the complementary region of the target sequence and the resulting opening of the loop is the fluorescence quenching canceled and a fluorescence signal increase generated. This makes it possible to detect the light energy of the fluorescence donor.

TaqMan probes also consist of an oligonucleotide and two terminal fluorescence molecules (Heid et al. 1996). However, the oligonucleotide does not form a loop, but the fluorescence is transmitted via spatial proximity to the oligonucleotide. When the probe is attached to the target gene and a primer is extended, the probe is degraded by the exonuclease activity of the polymerase used and thus the fluorophore is separated from the quencher. If the fluorescence quenching is too low due to a particularly long probe sequence and the associated large spatial separation of the fluorescence molecules, a further internal or terminal quencher can be attached. However, this is more challenging as the exact length of the cleaved sequences is often unknown and it is therefore possible that the cleaved sequence with fluorescence donor also contains one of the internal quenchers. In digital PCR, Taqman probe systems are also used for so-called “intensity multiplexing”. Here, Taqman probes with identical fluorescence labeling but different DNA sequences are used to differentiate a plurality of target sequences in a multiplex PCR. This is made possible by using the different types of TaqMan probes in different concentrations. However, this method also requires complex fine-tuning of the concentrations and is usually not very precise (Whale et al. 2016).

The modular reporter complex described in this invention has the advantage that different fluorescence intensities can be set at constant concentrations of detection molecule complexes by forming complexes from a plurality of fluorophore-labeled and quencher-labeled oligonucleotides. This requires less fine-tuning and enables more precise signal adjustment.

In addition to direct optical signal generation using a fluorescently labeled DNA probe, there are also systems in which detection is carried out using a DNA probe that is not fluorescently labeled. These also bind in a target sequence-specific manner and are cleaved by the exonuclease activity of a polymerase. In contrast to the previously described probe types, no fluorescence signal is generated during this cleavage, but the signal generation is initiated on a second molecule, which itself is independent of the target sequence. This has already been demonstrated in both PCR and LAMP (Faltin et al. 2012; Faltin et al. 2013).

One of these methods is known as mediator probe PCR, for which a patent application was filed by the University of Freiburg in 2012. In this system, two oligonucleotides are used for detection in the PCR instead of just one. These are referred to as the mediator probe (MP) and the universal reporter (UR). During PCR amplification of the target sequence, only a part of the one mediator probe binds sequence-specifically and is cleaved by the exonuclease activity of the polymerase. A second part of the mediator probe, the mediator that has not previously bound to the sequence, is separated. If no target sequence is present, the mediator probe remains intact. Once the mediator has been detached, it will bind to a universal reporter. A universal reporter is independent of the target sequence and, in addition to the mediator binding site, also has a fluorophore modification and quencher modification as well as a conformation that brings both modifications into spatial proximity to each other. By extending the mediator at the universal reporter, this spatial proximity is removed, e.g. by splitting off the fluorophore, resulting in a fluorescence signal of a specific wavelength. The advantage of this procedure is that it separates probe binding to the DNA sequence and fluorescence signal generation. This allows clear guidelines for mediator probe design to be established, universal reporters can be optimized once and then used for multiple sequences, and this two-part process also provides a double control, which makes signal generation very specific (Lehnert et al. 2018; Wadle et al. 2016, Schlenker et al 2021). However, one disadvantage of this technology is that the corresponding universal reporters are very complex and expensive to synthesize, as some of these modifications have to be made internally in the DNA sequence. This makes further development of these molecules with regard to imparting new properties very complex and difficult. In addition, further protective groups are required on a universal reporter.

Another shortcoming of this technology is that the fluorescence ranges of a color channel differ depending on the device, meaning that a separate fluorophore quencher optimization must be carried out for each device. This optimization is very complex and cost-intensive, as a new universal reporter has to be synthesized for each combination. In addition, the structure of the oligonucleotide is restricted, such that only a few options can be implemented for adding further labels for stronger signals or multiplex variations. This severely restricts the flexibility of the reporter with regard to testing different labels. The disadvantage of this system is therefore the very expensive production of the universal reporter due to its multiple modifications and the inflexible design.

During the same period, a technology was developed by Seegene Inc. which is also based on the separation of DNA sequence detection and signal generation via two detection molecules in real-time PCR. Here, an unlabeled PTO probe binds to a DNA target sequence, is cleaved and releases a fragment, which then forms an extended duplex with a fluorescently labeled target sequence-unspecific detection molecule (CTO molecule). This process is used to influence the signal generation over different lengths of these detection molecules. This makes it possible, for example, to differentiate between multiple target sequences in the same detection channel by reading the signal at defined, predetermined temperatures. Similar to a universal reporter, a CTO is also a single molecule that places corresponding requirements on the synthesis.

Another patent also uses readout at different temperatures in one approach and can therefore be interpreted as a further development of the Seegene technology mentioned above (PCT/CN2018/084794). Here, too, an extended reporter molecule is melted after detection. In contrast to the previous patent, a reporter molecule can be used to differentiate between different target sequences.

Patent WO2013079307A1 Bifunctional oligonucleotide probe for universal real-time multianalyte detection claims the mediator probe technology system. In this system, a mediator probe is activated by extending a primer (auxiliary molecule 1) by means of a polymerase (auxiliary molecule 2) at the target sequence (target molecule). Due to the exonuclease activity of the polymerase, the mediator probe is cleaved and can then bind to the universal reporter (UR) (mediator hybridization sequence). Here it acts as a primer after cleavage. As a result, it is extended by the polymerase and thus separates the fluorophore and quencher on the universal reporter.

Patent WO2018114674A1, regarding the loop-mediated isothermal amplification method with mediator-displacement probes (MD LAMP), claims a universal reporter with at least one oligonucleotide and at least one fluorophore and quencher for a LAMP reaction. Until now, it was assumed that this only works because a LAMP, unlike a PCR, has a continuously uniform temperature, which quickly establishes and maintains an equilibrium, allowing individual molecules to bind to each other permanently and thus not generate a signal in the initial state.

In the general prior art in science and technology, different wavelengths are used in optical detection reactions in order to be able to distinguish individual target sequences in certain light ranges, the so-called detection channels, in a reaction. This restricts multiplexing, as only one target sequence can be detected per channel. The degree of multiplexing in most commercial devices is therefore limited to five or six channels.

An alternative approach to multiplexing is represented by patents with detection systems that are also based on a separation of signal generation and detection, such as the patent specification US20200087718A1 from Seegene for signal molecule-based detection using melting curve analysis. This patent claims the possibility of increasing the degree of multiplexing by using different lengths of the target sequence-unspecific signal molecule to generate different melting temperatures. A further divisional application (EP2708608) uses only the signal readout at predefined temperatures by way of contrast to the original patent, since the corresponding one-part signal molecules of different sequence lengths also show different fluorescence behavior at defined readout temperatures.

The modular reporter complex described in this invention has the advantage that in certain embodiments a plurality of target sequences can be detected in one detection channel independent of temperature. For this purpose, a plurality of fluorophores or quenchers are used on a signal initiation strand or base strand, which provide different fluorescence intensities and thus make fluorescence signals assignable within the same channel. As a result, detected target sequences can be differentiated based on the fluorescence intensity generated. This enables the simultaneous detection of at least two target sequences within one detection channel without complex temperature-dependent readout steps.

Another patent is the patent of the company Biorad (U.S. Pat. No. 9,921,154 B2), which has only been granted in the USA. This also claims the detection of a plurality of target sequences in identical detection channels in a digital PCR, but only describes sequence-specific hydrolysis probes labeled with fluorophore and quenchers or intercalating dyes for such detection. Other patents from Biorad also describe the differentiation of various target sequences, in some cases in one channel. However, according to current understanding, these can only be used effectively if there is significant over-crowding of the droplets.

### OBJECTIVE OF THE INVENTION

PCR is the gold standard method for amplifying individual DNA sequences and making them detectable. Either intercalating dyes or DNA probes are used to detect and quantify PCR products (DNA or cDNA in the case of RNA). However, intercalating dyes bind non-specifically to all double-stranded DNA molecules present, making direct sequence-specific detection in PCR impossible. DNA probes are signal-generating DNA sequences that are complementary to the respective sequence section of a PCR product and can therefore specifically detect and quantify it. This method is used in particular in real-time PCR and digital PCR. Currently, DNA probes either have a biochemical modification themselves to generate a signal in the presence of a DNA target sequence during PCR (e.g. Taqman probes) or activate a second detection molecule that generates a signal independently of the target sequence (e.g. mediator probes in combination with target sequence-unspecific universal reporters).

In the case of optical detection, e.g. via fluorescence, such detection molecules or detection molecule systems also have the disadvantage that they generally have to have multiple biochemical labels and modifications at once (e.g. fluorophore and quencher in the case of a fluorogenic Taqman probe, fluorophore, quencher and 3′-block group in the case of a target sequence-unspecific universal reporter), which makes them more complex and expensive to synthesize and therefore inflexible in their design. This is a major problem, especially in the development and optimization of DNA detection reactions, as only a few systems of detection molecules can be tested. In addition, the possibilities of detection molecules and thus detection methods are considerably limited as a result, as it is not possible to make any desired number of modifications to a single DNA sequence. However, since the bonds between DNA sequences are broken and closed multiple times during PCR, the prior art assumes that all biochemical modifications must always be bound to a DNA sequence in a fluorogenic DNA detection molecule. According to the current assumption in the prior art, this is the only way to ensure the initially required spatial proximity between fluorophore and quencher in order for the quencher to suppress the signal of the fluorophore until these molecules are spatially separated during the detection process. The molecular processes that lead to such signals are also not always uniform, as probes for signal generation can either be cleaved or unfolded, for example. However, this leads to inhomogeneous signal generation, which leads to less precise results.

This highlights the urgent need for a new type of detection process and signal molecule that overcomes these problems.

The inventors have determined that this objective should be achieved by a new type of modular detection molecule complex which is target sequence-unspecific, flexible in design and easy to optimize, while possessing the same performance characteristics as the current prior art describes for one-part detection molecules. Up to now, such a modular composition has failed in particular because it does not use covalent chemical bonds, which makes it unsuitable for PCR applications according to current assumptions of the prior art, since the detection molecules themselves would be separated during thermal cyclic heating.

### SUMMARY OF THE INVENTION

The objective according to the invention is achieved by the features of the independent claims. Advantageous embodiments of the invention are described in the dependent claims.

Thus, in one aspect, the invention relates to a method for detecting at least one target nucleic acid sequence, comprising the steps of:
a. Providing at least one target sequence-unspecific modular reporter complex comprising at least one label and
   at least two oligonucleotides, namely
      i. a basic strand, comprising
         1. at least one mediator binding site
         2. at least one signal oligo binding site
      ii. at least one signal oligo
   wherein the at least one signal oligo binding site of the base strand and the at least one signal oligo hybridize with each other but are not covalently linked and together form a signal complex,
b. Providing at least one mediator probe, wherein the mediator probe comprises an oligonucleotide having at least one probe sequence and at least one mediator sequence, wherein
   the at least one probe sequence has an affinity for at least one target nucleic acid sequence, and the at least one mediator sequence has an affinity for at least one mediator binding site on the base strand of the at least one target sequence-unspecific modular reporter complex,
c. PCR amplification of at least one nucleic acid sequence,
d. Binding a probe sequence of at least one mediator probe to the at least one target nucleic acid sequence,
e. Cleavage of the probe sequence of the at least one mediator probe bound to the at least one target nucleic acid sequence by a PCR polymerase with nuclease activity during PCR amplification, wherein the mediator sequence is released,
f. Binding of at least one released mediator sequence to a mediator binding site of the at least one target sequence-unspecific modular reporter complex,
g. Extension of the sequence of at least one mediator sequence bound to a mediator binding site by a PCR polymerase, wherein
   the binding of the hybridized at least one signal oligo binding site and the at least one signal oligo is broken, thereby initiating a signal change,
h. Detection of at least one signal change as evidence of the at least one target nucleic acid sequence.

In a preferred embodiment of the method according to the invention, the at least one label of the target sequence-unspecific modular reporter complex comprises at least one fluorophore and/or at least one quencher.

In embodiments, the present method is used to detect at least one nucleic acid sequence during a PCR reaction, wherein a mediator probe is cleaved during this reaction (by an exonuclease activity of the polymerase). The cleavage product is a mediator, which subsequently binds to a target sequence-unspecific modular reporter complex and initiates a signal change via a subsequent reaction, which serves to detect the DNA sequence. In this embodiment, the target sequence-unspecific modular reporter complex preferably consists of at least two oligonucleotides which are not covalently linked, wherein at least one oligonucleotide of this complex has at least one label which initiates a signal change and which preferably comprises at least one fluorophore and at least one quencher. In this embodiment, an oligonucleotide of the target sequence-unspecific modular reporter complex (base strand) preferably has at least one binding site for at least one mediator sequence (herein also referred to as receptor) and at least one binding site for a signal oligo. In this embodiment, the second oligonucleotide of the complex is the signal oligo, which binds to the base strand (and thus forms a signal complex). As long as the mediator probe is uncleaved, the target sequence-unspecific modular reporter complex remains in its ground state during the detection or readout process. By extending the mediator oligonucleotide along the base strand during

PCR, the target sequence-unspecific modular reporter complex is broken up at the signal complex in such a way that the label or labels on the base strand and/or on the signal oligo are separated from each other, thereby initiating a change in the signal to the ground state and thus a signal change, which serves to detect the DNA target sequence.

In some embodiments, steps d-h of the method according to the invention may be performed continuously in each cycle during PCR amplification, this is preferably the case when the PCR amplification is real-time PCR or qPCR. In other words, in embodiments of the method according to the invention, steps d-h are repeated during PCR amplification in each PCR cycle, wherein the PCR amplification is a real-time PCR or qPCR.

In other embodiments, steps d-g of the method according to the invention may occur during PCR amplification, with step h occurring subsequently, this is preferably the case when the PCR amplification is a digital PCR or endpoint analysis. In other words, in embodiments of the method according to the invention, steps d-g are repeated during PCR amplification in each PCR cycle, followed by step h, wherein the PCR amplification is a digital PCR or endpoint analysis. In the case of a digital PCR and/or endpoint analysis, the detection step h of the method according to the invention is preferably carried out separately, following the PCR amplification reaction.

The modular reporter system for target sequence-unspecific detection according to the invention differs from the previously known detection systems by the flexible use of different oligonucleotides with different labels and surprisingly has the same performance characteristics in a PCR as current one-part detection molecules. Above all, the modular structure provides various advantages such as the design being flexibly adaptable to specific device conditions, the uniformity of the signal generation reaction between different detection methods or the inexpensive, simple production as well as completely new multiplex detection methods. The system and its functionality have not yet been described in a patent or in the literature on nucleic acid detection in a PCR.

The core of the invention is the modular target sequence-independent modular reporter complex (see FIG. 2A for an example embodiment) of non-covalently linked oligonucleotides. This system has all the advantages of the various target sequence-specific detection systems as well as target sequence-unspecific detection molecules and combines them with an increased flexibility in the design of such detection molecules, leading to entirely new detection methods. Surprisingly, the system can be used in a PCR with cyclic temperature changes, and the associated melting of the DNA strands in each cycle, without any problems. The results of the present examples, which are also shown in FIGS. 6-8, provide evidence that the modular system according to the invention even surpasses the functionality of the prior art Universal Reporter (UR). The significantly cheaper production can be achieved with at least as good performance parameters. A further advantage of the system is the possibility of flexible adaptation of the universal probe according to the invention. Different fluorophores and quenchers can be favorably combined with each other and investigated, and multiplex reactions can be expanded. Different probes can be distinguished in one fluorescence channel by means of different labels. The invention thus provides a dynamic, inexpensive and universal detection system.

The modular system of the target sequence-independent modular reporter complex according to the invention consists of a base strand. This preferably has at least one binding site for a mediator (receptor complex) and at least one binding site for a signal initiation oligonucleotide (or “signal oligo” for short) and thus forms a signal complex (examples of embodiments comprising a signal complex and a receptor complex can be found, for example, in FIGS. 2, 4 and 5). The base strand and at least one signal oligo together form a complex (see e.g. FIG. 2B for an example embodiment). Preferably, at least one of these strands has a label which can detect a structural change in this complex. This structural change preferably represents the separation, displacement or cleavage, detachment or enzymatic digestion of the signal oligo from the base strand, which preferably leads to a signal change. In embodiments, the modular structure results from a plurality of possible signal initiation oligonucleotides (or signal oligonucleotides, “signal oligos” for short) as well as additional labels, which can be attached both to the signal oligonucleotides and to the base strand (see e.g. FIG. 2B, label positions L₁ to Lᵢ on the signal complex). The advantages of target gene-dependent systems of the prior art can be taken up by various possibilities for attaching cis- or trans-labels. Various example embodiments are shown in FIGS. 4 and 5. Due to the modular structure, a much higher number of labels per complex can be attached than is possible with current one-part detection molecules.

Thus, in embodiments of the method according to the invention, the base strand comprises at least one signal oligo binding site to which two or more signal oligos are hybridized, and wherein the two or more signal oligos and/or the base strand have one or more labels at the at least one signal oligo binding site.

This novelty of the modular detection complex opens up new possibilities for detection reactions based on a basic reaction which is described below using the example of optical detection (see FIGS. 1 and 2A for an example embodiment):

In one embodiment, a mediator probe binds to the amplified DNA target sequence during a PCR detection reaction. A mediator probe is preferably an oligonucleotide and has a sequence-specific probe segment, which binds to the target sequence and is protected at the 3′-, and a target sequence-unspecific segment, called mediator, which does not bind to the target sequence except for a nucleotide common to mediator and probe. During primer extension by a polymerase with exonuclease activity (e.g. as part of an amplification reaction), the mediator is cleaved from the probe, leaving the common base on the mediator (FIG. 1). The mediator is now no longer blocked by the probe segment and can now bind to the target sequence-unspecific reporter complex and be extended here. Here, the mediator binds to the receptor complex of the base strand of the target sequence-independent modular reporter complex. The mediator is then extended along the base strand by the polymerase, wherein the signal complex is broken up in such a way that individual components and/or molecules of this complex are cleaved off, thereby initiating a signal change compared to the original state (FIG. 2A).

Thus, in some preferred embodiments, a mediator probe comprises an oligonucleotide and a sequence-specific probe segment that binds to the target sequence and is protected at the 3′ end. This protection at the 3′ end may be a block group (protecting group), e.g., a chemical block group or protecting group, which in some embodiments comprises a chain of three carbon atoms. Protection of the mediator probe at the 3′ end preferably prevents (unspecific) extension of the sequence strand by a polymerase during an amplification reaction. In accordance with the invention, in embodiments, the mediator probe may comprise any protecting or blocking group suitable for preventing (unspecific) extension of the mediator probe sequence strand by a polymerase during an amplification reaction. In some embodiments, the mediator probe is protected against (unspecific) polymerase extension by means other than a block group (protecting group) at the 3′ end.

In other embodiments, a mediator probe does not comprise a block group (protecting group) at the 3′ end and is not protected against (unspecific) polymerase extension.

In embodiments, a mediator probe protected at the 3′ end may comprise a “C3 spacer”. Such a C3 spacer may be a chemical block group, which in some embodiments comprises a chain of three carbon atoms. This “C3 spacer” thus preferably prevents (unspecific) polymerase extension of the mediator probe sequence strand. The person skilled in the art is familiar with typical and, depending on the embodiments, suitable block groups (protective groups). Also, based on the present disclosure of the invention, the person skilled in the art knows how to select suitable block groups (protecting groups) as routine adaptations of the invention described herein.

In preferred embodiments of the method according to the invention, no fluorescence signal change is generated by the at least one fluorophore when the at least one signal oligo is hybridized to the at least one signal oligo binding site of the base strand, wherein either the at least one quencher is localized at the at least one signal oligo binding site of the base strand and the at least one fluorophore is localized at the at least one signal oligo or vice versa, and wherein in step g at least one fluorophore and at least one quencher are separated, thereby initiating a signal change.

In the context of the invention, a fluorescence signal change preferably describes a significant, differentiable and/or characteristic change in the fluorescence signal which is clearly distinguishable or differentiated from potential base or background signals or background noise. Therefore, a fluorescence signal change in the context of the invention preferably describes a significant, differentiable and/or characteristic change in the fluorescence signal, and not a fluorescence base or background signal or background noise.

In embodiments, the at least one label further comprises at least one fluorophore and at least one quencher, wherein both the at least one quencher and the at least one fluorophore are localized on the at least one signal oligo, and wherein in step g. (extension of the sequence of at least one mediator sequence bound to a mediator binding site by a PCR polymerase), the at least one signal oligo is cleaved off by the PCR polymerase, whereby the at least one fluorophore and the at least one quencher are separated, thereby initiating a signal change. In embodiments, cleavage of the signal oligo from the signal oligo binding site of the base strand by a PCR polymerase can be accomplished either by enzymatic digestion or cleavage of the signal oligo by the polymerase (e.g., by exonuclease activity of the polymerase) or by another mechanism, e.g., by detaching, separating or displacing the signal oligo from the base strand by means of the polymerase.

The process according to the invention for detecting DNA sequences by means of PCR in combination with optical readout comprises a novel system of individual oligonucleotides, preferably DNA oligonucleotides. These form such a target sequence-independent modular reporter complex without covalent bonds. Surprisingly, this target sequence-independent modular reporter complex has all the advantages and performance characteristics of one-part target sequence-dependent DNA probes or one-part target sequence-independent detection molecules.

The target sequence-independent modular reporter complex preferably consists of at least two DNA sequences that specifically bind to each other and carry chemical modifications that initiate signal generation during a PCR reaction (e.g. DNA amplification). In some embodiments, by using different target sequence-independent reporter complexes with different numbers of labels and/or different numbers of signal oligos, different strength signals can be generated which make the activation of these target sequence-independent reporter complexes distinguishable. Thus, by combining multiple or different labels (e.g. different color and/or intensity), signals can be generated that can be distinguished from each other. For example, the signal of a signal oligo with one red label can be distinguished from the signal of a signal oligo with two or three red labels on the basis of the differences in intensity of the generated signal, or from the signal of a signal oligo with one red and one green label in terms of color. In embodiments of the present invention, not only individual labels are provided, but also different combinations of different fluorophore colors and/or the number of fluorophores (signal intensity), each encoding a signal that is specific for a target sequence. This combinability of signals is advantageous for the detection of a plurality of target sequences at the same time (in the same PCR reaction).

Multiplex detection reactions, i.e. simultaneous detection of different target sequences in a sample and in a reaction, require in the prior art either the availability of a plurality of optical channels, further process steps or complex concentration coordination of the reporter molecules. In contrast, the target sequence-independent modular reporter complex according to the invention enables combined detection via different channels, for which a base strand with more than one receptor complex can be used. In these embodiments, multiple receptor complexes (≥2) are staggered along the base strand so that they can activate different signal complexes (see FIG. 3 for an example embodiment). In various embodiments, a receptor complex can regulate or activate one or more signal complexes, preferably located upstream (towards the 5′ end).

Thus, in embodiments of the method according to the invention, the base strand comprises at least one signal oligo binding site to which two or more signal oligos are hybridized, and wherein the two or more signal oligos and/or the base strand have one or more labels at the at least one signal oligo binding site.

In some embodiments, the base strand comprises two or more signal oligo binding sites, and wherein at least one of the signal oligos hybridized to the two or more signal oligo binding sites and/or the base strand has one or more labels at at least one of the two or more signal oligo binding sites.

In some embodiments, the at least one target sequence-unspecific modular reporter complex enables detection of at least a first and a second target nucleic acid sequence, wherein the base strand comprises
at least a first and a second mediator binding site for at least a first and a second mediator sequence of at least a first and a second mediator probe, and at least a first and a second signal oligo binding site to which at least a first and a second signal oligo is hybridized, and
wherein the first mediator probe comprises a probe sequence having an affinity for a first target nucleic acid sequence and the second mediator probe comprises a probe sequence having an affinity for a second target nucleic acid sequence.

In some of these embodiments, the base strand comprises at least a first label and a second label, wherein the signal change due to the at least one first label is characteristic of the first target nucleic acid sequence, and the signal change due to the at least one second label is characteristic of the second target nucleic acid sequence.

In some embodiments, in step a. at least a first and a second target sequence-unspecific modular reporter complex is provided, wherein the at least first target sequence-unspecific modular reporter complex enables the detection of at least a first target nucleic acid sequence and the at least second target sequence-unspecific modular reporter complex enables the detection of a second target nucleic acid sequence, wherein the signal change due to the at least one label of the at least first target sequence-unspecific modular reporter complex is characteristic of the first target nucleic acid sequence and the signal change due to the at least one label of the at least second target sequence-unspecific modular reporter complex is characteristic of the second target nucleic acid sequence.

Preferably, different extended mediator sequences can be encoded by different color combinations so that different target sequences can be detected. Thus, a target sequence-independent modular reporter complex according to the invention is flexible in design.

Therefore, in some embodiments, the signal changes characteristic of the at least first target nucleic acid sequence and the signal changes characteristic of the at least second target nucleic acid sequence differ from each other by their color and/or their fluorescence or signal strength.

In embodiments, different encodings of fluorescent signals may be used. In one embodiment, a signal complex comprises at least two signal oligos with two labels, wherein the common signal is characteristic of a target sequence.

Thus, in embodiments, it is possible to combine a plurality of signal oligos with at least one label each in order to generate a signal specific to a common target sequence. By combining a plurality of labels of the same or different colors, signals can be generated that differ from other signals for other target sequences by their color and/or their intensity. Mixed colors can also be generated by combining different fluorescent colors of labels and optionally also by means of their number.

In another embodiment, the base strand comprises at least two signal complexes with different signal oligos and/or different labels on the signal oligos, wherein the base strand comprises a single receptor complex corresponding to the at least two signal complexes with at least one mediator binding site, e.g. downstream (towards the 3′ end) of the signal complexes. In this embodiment, a target sequence may be encoded by two colors.

The achieved effect of these embodiments is a colorimetrically encoded multiplexing in the digital PCR or qPCR detection or amplification reaction, which enables the detection of more (a larger number of) target sequences than detection channels are available. An example of color coding of fluorophores would be the signal combinations: Target sequence 1: red-red, target sequence 2: red-green, target sequence 3: green-green, target sequence 4: green-green-red.

The detection of a plurality of target sequences within a wavelength range can be carried out by a quantitative observation of the fluorescence values or combined with other parameters (e.g. the readout temperature). The signal generation can be influenced by different lengths of the signal oligos so that a plurality of target sequences can be differentiated in the same detection channel. For this purpose, specific readout temperatures for signal detection must be defined before the analysis. Depending on the selected parameters, such as the length of the signal oligo and/or the selected fluorophores, this results in a different signal at different temperatures. By iteratively reading out the fluorescence signal at different temperatures, a specific signal change can be detected at certain temperature ranges, which is characteristic of the selected fluorescence modifications or lengths of the signal oligos. This allows conclusions to be drawn as to which modular reporter complex has been activated.

Therefore, in some embodiments, the detection of the signal change comprises an analysis of the signal change as a function of the detection temperature.

The method according to the invention described here has the advantage over the multiplexing method described in U.S. Pat. No. 9,921,154 B2 that the method according to the invention can also be used without multiple occupancy of each reaction space and/or a set of different types of modular reporter complexes can generate fluorescence signals of different levels by multiple labeling via, for example, in this case very easily attached single or multiple signal oligos, without the need for complex synthesis. Without the use of multiple-labeled target sequence-specific hydrolysis probes that are complex to synthesize, as described in the method of U.S. Pat. No. 9,921,154 B2, this method can only be used according to current understanding if a significant multiple occupancy with DNA target sequences occurs per reaction space of a digital PCR, which thus lead to distinguishable signal clusters in the data space.

In addition, a target sequence-independent modular reporter complex according to the invention has a very high stability under PCR conditions in the initial state. Accordingly, the initial signal is comparable to that generated by a one-part DNA probe or a one-part target sequence-independent reporter. As a result, the overall resulting PCR detection system according to the invention has comparable performance characteristics to current PCR detection methods based on one-part detection molecules. This is the case even though this complex of a target sequence-independent modular reporter has to be separated with each cycle of a PCR and formed again before the signal readout.

The system according to the invention thus increases the efficiency of signal generation and is also suitable for the simultaneous detection of a plurality of DNA target genes in a PCR reaction (multiplex PCR). In addition, it offers completely new possibilities for detecting and differentiating a plurality of DNA sequences in the same channel of a PCR detector.

In embodiments of the method according to the invention, steps c. to h. are carried out as part of a reaction selected from the group comprising PCR, digital PCR, RT-PCR, digital RT-PCR, real-time/qPCR, droplet PCR, or any combination thereof.

In one embodiment of the method according to the invention, steps c. to h. are carried out as part of a PCR reaction, preferably a digital PCR reaction.

In other embodiments of the method according to the invention, steps c. to h. are carried out as part of a PCR reaction, preferably a real-time PCR or qPCR reaction.

In some of the above-mentioned embodiments of the method according to the invention, steps c. to h. are carried out as part of a droplet PCR or emulsion PCR reaction.

These different applications are possible because the target sequence-independent modular reporter complex according to the invention is both flexible in design and the individual components are inexpensive to develop and produce. In addition, the system according to the invention enables the production of universal microarrays. Overall, this represents a significant improvement over the prior art.

In a further aspect, the invention relates to a kit for carrying out the method according to any one of the preceding claims comprising:
at least one oligonucleotide primer
at least one mediator probe
at least one signal oligo
at least one basic strand
at least one buffer
PCR polymerase.

In the context of the kit according to the invention, the oligonucleotide primers, preferably at least one oligonucleotide primer pair, the at least one signal oligo, the at least one mediator and/or the at least one base strand may be configured or suitable for the specific detection of one or more different target sequences.

In embodiments, the kit according to the invention can be used for carrying out the method according to the invention.

In some embodiments, the kit can thus be used for the specific amplification and/or detection of target sequences within the framework of the method according to the invention. In preferred embodiments of the use of the kit according to the invention, the specific amplification and/or detection reaction is a PCR, qPCR, real-time PCR, droplet PCR and/or digital PCR.

The embodiments described for one aspect of the invention may also be embodiments of any of the other aspects of the present invention. Accordingly, embodiments described for the method according to the invention may also be embodiments of the kit according to the invention. Furthermore, any embodiment described herein may also comprise features of any other embodiment of the invention. The various aspects of the invention are unified by, benefit from, are based on, and/or are related to the common and surprising discovery of the unexpected beneficial effects of the present method, namely optimized PCR detection of target sequences by reporter complexes which are themselves target sequence-unspecific.

### DETAILED DESCRIPTION OF THE INVENTION

The term “target sequence-unspecific reporter complex” describes a complex of target sequence-unspecific nucleic acid oligonucleotides (e.g. DNA oligonucleotides) for signal generation during a PCR in the presence of DNA target sequences. Preferably, a target sequence-unspecific reporter complex comprises at least one label, and at least two oligonucleotides, namely 1) a base strand comprising at least one mediator binding site and at least one signal oligo binding site, and 2) at least one signal oligo, wherein the signal oligo binding site of the base strand and the at least one signal oligo hybridize to each other but are not covalently linked.

In the context of the present invention, the term “base strand” describes a nucleic acid oligonucleotide (e.g. a DNA oligonucleotide) and part of the target sequence-unspecific reporter complex. A base strand serves as a basis for the binding of signal oligos and mediators, which thus form a signal complex and receptor complex. Therefore, a base strand preferably comprises at least one mediator binding site and at least one signal oligo binding site. In embodiments, a base strand comprises one or more mediator binding sites and/or signal oligo binding sites. Thus, a base strand may comprise at least 1, 2, 3, 4, 5, 6, 7, 8, 9, 10, 11, 12, 13, 14, 15, 16, 17, 18, 19, 20, 25, 30, 40 or even 50 mediator binding sites. A base strand may further comprise at least 1, 2, 3, 4, 5, 6, 7, 8, 9, 10, 11, 12, 13, 14, 15, 16, 17, 18, 19, 20, 25, 30, 40 or even 50 signal oligo binding sites. In preferred embodiments, a base strand comprises between 1 and 10 mediator binding sites and between 1 and 10 signal oligo binding sites. A mediator binding site may correspond to one or more signal oligo binding sites, in other words, an (activated) mediator bound to a mediator binding site may, when extended by a (PCR) polymerase, lead to the activation or degradation, digestion, cleavage or release of one or more signal oligos from one or more, preferably upstream (towards the 5′ end), signal oligo binding sites. A base strand may comprise one or more signal complexes and one or more receptor complexes, wherein a signal oligo complex may comprise at least one signal oligo binding site and (in the non-activated state) at least one signal oligo, and a receptor complex may comprise at least one mediator binding site.

In the context of the present invention, a “signal initiation oligonucleotide”, “signal initiation oligo” or “signal oligo” for short is a nucleic acid oligonucleotide (preferably a DNA oligonucleotide) and part of the signal complex which, in the presence of a target sequence, initiates a signal change by itself and or parts thereof being cleaved or broken up from the signal complex. In the context of the present invention, the terms signal initiation oligonucleotide, signal initiation oligo and signal oligo are to be regarded as equivalent and interchangeable.

In the context of the present invention, a “label” may describe one or more fluorophores or one or more quenchers. Accordingly, in the context of the invention, in embodiments, a base strand and/or a signal initiation molecule or signal oligo may carry or comprise one or more fluorophores and/or quenchers. Proximity of a fluorophore to the quencher prevents detection of its fluorescence, wherein degradation of the signal oligo by hydrolysis by the 5′-to-3′-exonuclease activity of the PCR polymerase used for the amplification reaction disrupts the reporter-quencher proximity, allowing unquenched emission of fluorescence that can be detected upon excitation with a laser. In preferred embodiments, a target sequence-unspecific modular reporter complex comprises at least one fluorophore and at least one quencher (thus these are preferably present “in pairs”), wherein the quencher preferably suppresses the fluorophore signal as long as the signal oligo is hybridized at the signal oligo binding site, wherein the at least one fluorophore and the at least one quencher may be located relative to each other within the target sequence-unspecific modular reporter complex either in cis-position (both located either at the signal oligo or at the base strand) or in transposition (one of the two labels is located at the base strand, the other at the signal oligo, or vice versa). In embodiments in which a plurality of quencher-fluorophore pairs are present within a target sequence-unspecific modular reporter complex, the pairs may also be arranged in different localizations relative to each other, e.g., some in cis and others in trans, or all in cis or trans. A base strand may comprise none, one or more labels, for example there may be none or at least 1, 2, 3, 4, 5, 6, 7, 8, 9, 10, 11, 12, 13, 14, 15, 16, 17, 18, 19, or 20 labels, or exactly 0, 1, 2, 3, 4, 5, 6, 7, 8, 9, 10, 11, 12, 13, 14, 15, 16, 17, 18, 19, 20 or even 25 labels. In some embodiments, a base strand comprises none, 1, 2, 3 or up to 5 labels. In other embodiments, a base strand comprises none, 1, 2, 3, 4, 5, or even more than 5 labels. A signal oligo may comprise none, one or more labels, for example there may be none or at least 1, 2, 3, 4, 5, 6, 7, 8, 9, 10, 15, or even 20 labels, or exactly 0, 1, 2, 3, 4, 5, 6, 7, 8, 9, 10, 11, 12, 13, 14, 15, 16, 17, 18, 19, 20 labels. In some embodiments, a signal oligo comprises none, 1, 2, 3 or up to 5 labels. In other embodiments, a signal oligo comprises none, 1, 2, 3, 4, 5, or even more than 5 labels.

Herein, a “mediator” refers to an oligonucleotide and part of the receptor complex which can be extended along the “base strand” by the polymerase. A “mediator probe” describes a nucleic acid oligonucleotide, preferably a DNA oligonucleotide, which establishes the connection between the target sequence and the target sequence-independent (modular) receptor by binding to a DNA target sequence during a PCR in the presence of the target sequence, being cleaved by the exonuclease activity of the polymerase and releasing a mediator sequence which then binds to a base strand and forms a receptor complex together with the base strand.

For the purposes of the invention, a “C3 spacer” is preferably a chemical block group (protecting group), which preferably comprises a chain of three carbon atoms. This block group (protecting group) preferably serves to prevent (unspecific) polymerase extension of the strand. The person skilled in the art is familiar with typical and-depending on the embodiments-suitable C3 spacers/block groups (protecting groups) and the person skilled in the art knows, based on the present disclosure of the invention, how to select suitable C3 spacers/block groups (protecting groups) as routine adaptations of the invention described herein.

The term “nucleic acid” refers to nucleic acid molecules including, without limitation, DNA, SSDNA, dsDNA, RNA, mRNA, tRNA, lncRNA, ncRNA, microRNA, siRNA, rRNA, sgRNA, piRNA, rmRNA, snRNA, snoRNA, scaRNA, gRNA, or viral RNA. Nucleic acid sequences herein refer to a consecutive arrangement of nucleotides, where the nucleotides are represented by their nucleobases in guanine (G), adenine (A), cytosine (C) and thymine (T) in DNA and uracil (U) in RNA. A nucleic acid sequence herein may also refer to the sequence of consecutive letters or nucleobases (consisting of G, A, C and T or U) representing the actual sequence of consecutive nucleic acids in a DNA or RNA strand. This nucleic acid sequence can be identified and characterized biochemically and bioinformatically using DNA or RNA sequencing or specifically detected by complementary nucleic acid probes (e.g., in embodiments herein by mediator probes), e.g., as part of a PCR, real-time PCR or detection reaction of a digital PCR. The sequence analysis may also comprise comparing the nucleic acid sequence obtained or a detection signal specific thereto with one or more reference nucleic acid sequences and/or with the detection signals of housekeeping genes. The term nucleotide may be abbreviated as “nt”. The term base pair (two nucleobases bonded to each other via hydrogen bonds) may be abbreviated as “bp”. In the context of the invention, a “target sequence” describes any nucleic acid sequence of interest which is to be detected by the method according to the invention. A target sequence may preferably be a DNA or RNA sequence. A target sequence may represent a part or the entire nucleic acid sequence of a target DNA. A mediator probe preferably comprises a sequence which is wholly or partially complementary to the nucleic acid sequence of the target sequence or a segment thereof. In some embodiments, this mediator probe sequence is 100%, 99%, 95%, 90% or 80% complementary to the target sequence. In some embodiments, a mediator probe can tolerate one or more mismatches to the target sequence and still bind to it. In other embodiments, the mediator probe only binds to a target sequence if it is 100% complementary to the target sequence.

The term “nucleic acid amplification reaction” refers to any process comprising an enzymatic reaction that enables the amplification of nucleic acids. A preferred embodiment of the invention relates to a polymerase chain reaction (PCR). “Polymerase chain reaction” (“PCR”) is the gold standard method for rapidly producing millions to billions of copies (full copies or partial copies) of a given DNA sample, enabling amplification of a very small DNA sample to a sufficiently large amount. PCR amplifies a specific region of a DNA strand (the DNA target sequence) depending on where the used primers bind to start the amplification reaction. Almost all PCR applications use a heat-stable DNA polymerase enzyme, such as Taq polymerase. Quantitative PCR (“qPCR”), or “real-time PCR”, is a specific form of PCR and is a standard method for detecting and quantifying a specific target sequence or quantifying gene expression levels in a sample in real time. In qPCR, fluorescently labeled probes or nucleic acids (e.g., mediator probes) are hybridized in the PCR reaction and, in embodiments, cleaved or digested by the PCR polymerase during primer extension once they bind to a complementary sequence (e.g., a target sequence), wherein, in embodiments, the presence and amplification of target sequences is monitored in real time after or during each PCR cycle. A real-time PCR allows the progress of an ongoing amplification reaction to be monitored as it occurs (i.e., in real time). Data is therefore collected throughout the PCR reaction and not at the endpoint as with conventional PCR. The measurement of reaction kinetics in the early stages of PCR offers significant advantages over conventional PCR detection. In embodiments of real-time PCR, reactions are characterized by the time during the cycle when amplification of a target is first detected, rather than by the amount of target accumulated after a fixed number of cycles, as in conventional PCR. The higher the starting copy number of the nucleic acid target, the more likely it is that a significant increase in fluorescence will be observed. Real-time PCR enables analysis by means of optical signals that are used to detect a specific PCR product (the target sequence) using specific fluorochromes or fluorophores. An increase in the DNA product during a PCR therefore leads to an increase in the fluorescence intensity measured at each cycle. Using different colored labels, fluorescent probes can be used in multiplex assays to monitor multiple target sequences.

While real-time qPCR is dependent on the relative amount of target nucleic acid being determined in each amplification cycle, “digital PCR” allows the absolute amount of target nucleic acid to be determined on the basis of Poisson statistics, which are used to calculate the amount of target nucleic acid following endpoint PCR amplification. The steps prior to amplification are usually comparable or similar between digital PCR and qPCR. However, in qPCR preferably all nucleic acid molecules are pooled and subsequently amplified and analyzed, whereas in digital PCR the nucleic acid molecules are preferably partitioned as best as possible into individual partitions (e.g. emulsion droplets, wells or gel beads), allowing the PCR to proceed as a single reaction in each partition (in the case of emulsion droplets, this reaction is also often referred to as droplet PCR or digital droplet PCR) and allowing separate analysis of each partition. In digital PCR, the random division of the nucleic acid molecules into individual partitions takes place according to the Poisson distribution. When analyzing digital PCR, Poisson statistics are then applied to determine the average number of nucleic acid molecules per partition (none, one or more). Poisson statistical analysis of the number of positive and negative reactions provides a precise absolute quantification of the target sequence.

The specificity of the mediator probes also prevents interference of the measurements by primer dimers, which are undesirable potential by-products in PCR. In one embodiment, the invention relates to a method wherein the amplification is a multiplex PCR with more than one primer pair. Multiplex PCR is a variant of standard PCR in which two or more target sequences can be amplified and/or detected simultaneously in the same reaction by using at least one primer pair in the reaction.

In the context of the invention, a “signal change” describes a fluorescence signal change. This signal change is preferably a significant, differentiable and/or characteristic change in the fluorescence signal which is clearly distinguishable or differentiated from potential base signals or background signals, or base noise or background noise. The person skilled in the art is aware that under some test conditions in the context of fluorescence detection, unspecific fluorescence base signals or background noise can occur due to fluorophores. Therefore, a signal change in the context of the invention preferably describes a significant, differentiable and/or characteristic change in the fluorescence signal, and not a fluorescence base signal or background signal or background noise. In preferred embodiments, this signal change can mean an increase in fluorescence intensity, in other words an increase in the fluorescence signal. In some embodiments, a signal change is a decrease in the fluorescence signal. The increase of a fluorescence signal is preferably due to the fact that an amplification reaction increases the number of target sequence amplicons and thereby the activation of associated signal complexes. Accordingly, the number of resulting cleavages, digestions and/or separations of the respective signal oligos from their binding site on the associated base strands increases, whereby at least one fluorophore is released and/or separated from its quencher (i.e. the distance between quencher and fluorophore increases such that the fluorescence signal is no longer quenched by the quencher). An increase (increase in the number) of released and/or non-quenched fluorophores thus leads to an increase in the fluorescence signal, which is specific and indicative of a target sequence. The more target sequences are thus present and are bound by mediator probes in a PCR reaction, the more the fluorescence signal increases. Preferably, the fluorescence signal is proportional or approximately proportional to the amount of the corresponding target sequence for which the fluorophore signal (e.g. its color) is specific/characteristic. Since in the context of a digital or “droplet” PCR preferably only one target sequence is present per reaction space (e.g. partition, emulsion droplet), the signal increases with the number of target sequence amplicons per reaction space. In preferred embodiments, there is ideally a uniform distribution of max. 1 target sequence per reaction space (e.g. partition, emulsion droplet), so that when amplification begins and a similar amplification efficiency is present in all reaction spaces (containing a target sequence), the specific signal for the detection of an identical target sequence in different reaction spaces is generated with a comparable amount/strength/intensity during readout by digital or “droplet” PCR, which is preferably indicative of the presence and/or number of target sequence amplicons present in each reaction space. In embodiments, the intensity/strength of a respective label, preferably specific to a target sequence, as well as the maximum achievable signal strength/intensity may depend on the number of labels per signal oligo and signal complex and/or the type of label (e.g. type of fluorophores and/or quencher).

“Fluorophore” (or fluorochrome, similar to a chromophore) is a fluorescent chemical compound capable of re-emitting light upon light excitation. Fluorophores for use as labels in the design of labeled probes of the invention include, without claiming to be exhaustive, rhodamine and derivatives such as Texas Red, fluorescein and derivatives such as 5-bromomethylfluorescein, Lucifer Yellow, IAEDANS, 7-Me2N-coumarin-4-acetate, 7-OH-4-CH3-coumarin-3-acetate, monobromobimane, pyrene trisulfonates such as Cascade Blue and monobromotrimethyl ammoniobimane, 7-NH2-4CH3-25-coumarin-3-acetate (AMCA), FAM, TET, CAL Fluor Gold 540, JOE, VIC, Quasar 570, CAL Fluor Orange 560, Cy3, NED, Oyster 556, TMR, CAL Fluor Red 590, HEX, ROX, LC Red 610, CAL Fluor Red 610, Texas Red, LC Red 610, CAL Fluor Red 610, LC Red 640, CAL Fluor Red 635, Cy5, LC Red 670, Quasar 670, Oyster 645, LC Red 705, Cy5.5, BODIPY FL, Rhodamine Green, Oregon Green 30 488, Oregon Green 514, Cal Gold, BODIPY R6Gj, Yakima Yellow, Cal Orange, BODIPY TMR-X, JOE, HEX, Quasar-570/Cy3, TAMRA, Rhodamine Red-X, Redmond Red, BODIPY 581/591, Cy3.5, Cal Red/Texas Red, BODIPY TR-X, BODIPY 630/665-X, Quasar-670/Cy5, Pulsar-650, Dy490, Atto-488, Atto532, Atto-Rho-6G, Dy590, Atto-Rho101, Cy5, Dy-636, Atto-647N, Cy5.5, Dy682, Atto-680, BMN-488, BMN-505, BMN-536, BMN-562,

“Quenching refers to any process that reduces the fluorescence intensity of a given substance. Quenching is the basis for Forster resonance energy transfer (FRET) assays or static or contact quenching assays or a combination of both. FRET is a dynamic quenching mechanism, as the energy transfer takes place while the donor is in an excited state. Contact quenching requires close proximity in the form of physical contact between donor and quencher. A quencher is a molecule that quenches the fluorescence emitted by the fluorophore when it is excited by the light source of a PCR cycler or detection device. Quenchers for use as labels in the construction of labeled signal oligos and/or base strands of the invention include, without claiming to be exhaustive, DDQ-I, Iowa Black, Iowa Black FQ, QSY-9, BHQ-1, QSY-7, BHQ-2, DDQ-II, 22 Eclipse, Iowa Black RQ, QSY-21, BHQ-3 Dabcyl, QSY-35, BHQ-0, ElleQuencher, BMN-Q1, BMN-Q2, BMN-Q60, BMN-Q-535, BMN-Q590, BMN-Q620, BMN-Q650. The person skilled in the art knows suitable pairs of reporter quenchers and knows which ones to select for a particular application.

### Embodiments

The invention is further described by the following examples. These are not intended to limit the scope of the invention, but represent preferred embodiments of various aspects of the invention, which are provided to illustrate the invention described herein.

### Description of the Methods and Implementation of the Embodiments

For the embodiments according to the results shown in FIG. 6, a real-time Mediator Probe PCR was performed on the Rotor-Gene Q 6000 6-plex from the manufacturer Qiagen. The temperature profile consists of an initial denaturation for 2 min at 95° C. and subsequent 45 cycles (1 cycle corresponds to: 10 seconds at 95° C., 80 seconds at 58° C. and a further 10 seconds at 58° C. for fluorescence readout).

Another embodiment comprises performing a digital PCR in the QIAcuity system from Qiagen according to the results shown in FIG. 7. The temperature profile results from an initial denaturation over a period of 2 min. This is followed by a 40-fold repetition of a cycle consisting of 15 seconds of denaturation at 95° C. and a subsequent binding step at 58° C. for 30 seconds.

In order to generate the results as shown in FIG. 9 of the multiplex variants embodiment, a digital multiplex Mediator Probe PCR can be performed using three different mediator probes for three different target sequences, wherein the following three complexes, each consisting of a base strand and one or two signal oligos, are used as modular reporter complexes for the three respective mediator probes:
Modular reporter complex 1: Sequence ID 11 (SEQ ID NO: 11), Sequence ID 12 (SEQ ID NO: 12)
Modular reporter complex 2: Sequence ID 13 (SEQ ID NO: 13), Sequence ID 14 (SEQ ID NO: 14)
Modular reporter complex 3: Sequence ID 15 (SEQ ID NO: 15), Sequence ID 11, Sequence ID 13

In embodiments, the digital PCR can be carried out analogously to the description of the test in the embodiment for digital PCR and to the procedure according to test 7, wherein a fluorescence signal must be measured in at least two suitable wavelength ranges in order to obtain and evaluate the signal patterns shown in Table 2.

Procedure for the test in FIG. 6: For a reaction volume of 25 μl per tube, the fluorescence in the green channel (wavelength: excitation at 470 nm, detection at 510 nm) was measured with optimized gain settings for NTC (without target) and PTC (with target) duplicates. Plasmid DNA Haemophilus ducreyi (short: Hd) was selected as target, of which 2.5 pg were added per single reaction (PTC). The primers used were purchased from Biomers.net GmbH, Ulm, Germany and are listed in Table 1. The PerfeCTa Multiplex qPCR ToughMix from Quantabio, Beverly, Massachusetts was used in single concentration. The forward and reverse primers for Hd [Sequence ID: 08 and 09] were used in 0.1 μM as well as 0.2 μM of the corresponding mediator probe [Sequence ID: 10].Within the test, the prior art Universal Reporter (UR) was compared with the two-part reporter and two multiplex variants were tested. The concentrations of the varying fluorescence-generating probes for the individual reactions were selected as follows: The UR (Sequence ID (SEQ ID NO): 01) was used in 0.05 μM, the two-part reporters consisting of signal oligo and base strand [Sequence ID (SEQ ID NO): 02 and 03] were used in 0.08 μM each. For the two multiplex variants, 0.05 μM of each of the two signal oligos [Sequence ID (SEQ ID NO): 05 and 06] and 0.1 μM of the base strand oligo [Sequence ID (SEQ ID NO): 04 and 07] were used. It is clear to the person skilled in the art how the test is to be carried out using the specified parameters, primer sequences and concentrations. This results in the curve shown in FIG. 6.

Procedure for the test in FIG. 7: The QIAcuity Probe Mix from Qiagen was used for the digital PCR in single concentration with an internal reference dye. The target and its concentration are identical to the example described above. The sequences are shown in Table 1. Of the forward and reverse primers, 0.8 μM each [Sequence ID: 08 and 09] were used as well as the mediator [Sequence ID (SEQ ID NO): 10] in 1.6 μM concentration. A concentration of 0.4 μM per reaction was selected for both the universal [Sequence ID (SEQ ID NO): 01] and the two-part reporters [Sequence ID (SEQ ID NO): 02 and 03]. A 26K 24-well nanoplate was filled with 40 μl reaction volume per well. The fluorescence was measured on the QIAcuity system in the green color spectrum (excitation: 463-503 nm, emission: 518-548 nm) and the standard settings were maintained, which are known to the person skilled in the art. The results shown in FIG. 7, which were evaluated using the QIAcuity Software Suite 1.2.18 by means of absolute quantification, were obtained for the test. A1 corresponds to the NTC, A2 to the PTC for the prior art (UR green, [Sequence ID (SEQ ID NO): 01]). Well C1 and C2 show the results of the NTC and PTC for the two-part reporter system [Sequence ID (SEQ ID NO): 02 and 03].

TABLE 1
Primer sequences used for the embodiments with designation and (internal)
modification. The sequences for the multiplex variants are only used
for the embodiment of the test in FIG. 6.

| Sequence | | | | |
|---|---|---|---|---|
| ID (SEQ | Sequence | | | Internal modification |
| ID NO) | (5′-3′) | 5′ Modification | 3′ Modification | fluorophore |

Universal reporter green (prior art)

| | | | | |
|---|---|---|---|---|
| 01 | ATTGCGGGA- | BMN-Q-535 | C3 spacer: | 8 = dT-FAM |
| | GATGAGACCCG- | | Chemical block | |
| | CAA8TGTT- | | group comprising | |
| | GGTCG- | | a chain of three | |
| | TAGAGCCCAGA | | carbon atoms to | |
| | ACGA | | prevent unspe- | |
| | | | cific polymerase | |
| | | | extension of the | |
| | | | strand. | |

Two-part reporter green

| | | | |
|---|---|---|---|
| 02 | GCGGGTCTCAT | | FAM |
| | CTCAC- | | |
| | GCTGATGTCT | | |
| 03 | AGA- | BMN-Q535 | C3 spacer: |
| | CATCAGCGTGA- | | Chemical block |
| | GATGAGACCCG- | | group comprising |
| | CAATGTT- | | a chain of three |
| | GGTCG- | | carbon atoms to |
| | TAGAGCCCAGA | | prevent unspe- |
| | ACGA | | cific polymerase |
| | | | extension of the |
| | | | strand. |

Multiplex variant 1

| | | | | |
|---|---|---|---|---|
| 04 | GACAGTCGTCG- | | C3 spacer: | 5 = BMN-Q-535 modifi- |
| | CATGCTG- | | Chemical block | cation of the sugar- |
| | CAGTG55GGA- | | group comprising | phosphate backbone |
| | GAG- | | a chain of three | between the two adja- |
| | CAGAAGTCCGT | | carbon atoms to | cent nucleotides |
| | GTGCATT- | | prevent unspe- | |
| | GGTCG- | | cific polymerase | |
| | TAGAGCCCAGA | | extension of the | |
| | ACGA | | strand. | |
| 05 | TGCACAC- | | FAM | |
| | GGACTTCTGCTC | | | |
| | TCC | | | |
| 06 | CACTGCAG- | FAM | | |
| | CATGCGAC- | | | |
| | GACTGTC | | | |

Multiplex variant 2

| | | | | |
|---|---|---|---|---|
| 07 | GACAGTCGTCG- | | C3 spacer: | 5 = BMN-Q-535 modifi- |
| | CATGCTG- | | Chemical block | cation of the sugar- |
| | CAGTG5GGA- | | group comprising | phosphate backbone |
| | GAG- | | a chain of three | between the two adja- |
| | CAGAAGTCCGT | | carbon atoms to | cent nucleotides |
| | GTGCATT- | | prevent unspe- | |
| | GGTCG- | | cific polymerase | |
| | TAGAGCCCAGA | | extension of the | |
| | ACGA | | strand. | |
| 05 | TGCACAC- | FAM | FAM | |
| | GGACTTCTGCTC | | | |
| | TCC | | | |
| 06 | CACTGCAG- | | | |
| | CATGCGAC- | | | |
| | GACTGTC | | | |

Primer Hd

| | |
|---|---|
| 08 | GTCGTCAGCTC |
| | GTGTTGTGA |
| 09 | TCCCCAC- |
| | CTTCCTCCAG- |
| | TTT |

Mediator probe sequence

| | |
|---|---|
| 10 | GGGCTCTAC- |
| | GACCAAATGTT- |
| | GGGTTAAGTCC |
| | CGCAACGAG |

TABLE 2
Primer sequences used with designation and (internal) modification for the
embodiment (multiplex detection) according to the method shown in FIG. 9.

| Se- | | | | |
|---|---|---|---|---|
| quence | | | | Internal |
| ID (SEQ | Sequence | | | modification |
| ID NO) | (5′-3′) | 5′ Modification | 3′ Modification | fluorophore |

Two-part reporter red (suitable for mediator with sequence ID: 16)

| | | | |
|---|---|---|---|
| 11 | GCCG- | | Cy5 |
| | CATCTAATGAGG | | |
| | TCGAGGAGTCA | | |
| 12 | AG- | BHQ-1 | C3 spacer: |
| | TGACTCCTCGAC- | | Chemical block |
| | CTCATT- | | group comprising |
| | AGATGCGG- | | a chain of three |
| | CATTCGATCACA | | carbon atoms to |
| | CAACATGAG- | | prevent unspe- |
| | CATGTGTAC | | cific polymerase |
| | | | extension of the |
| | | | strand. |

Two-part reporter green (suitable for
mediator with sequence ID (SEQ ID NO): 17)

| | | | |
|---|---|---|---|
| 13 | CCGCGCGTCTT- | | Atto 488 |
| | GGAGCAGTCCTT- | | |
| | GTT | | |
| 14 | AACAAGGACTGC | BMN-Q1 | C3 spacer: |
| | TCCAAGAC- | | Chemical block |
| | GCGCGGATTAG- | | group comprising |
| | CATGTGAG- | | a chain of three |
| | GAACAC- | | carbon atoms to |
| | GATGACAC | | prevent unspe- |
| | | | cific polymerase |
| | | | extension of the |
| | | | strand. |

Multiplex variant red and green (suitable for
mediator with sequence ID (SEQ ID NO): 18)

| | | | | |
|---|---|---|---|---|
| 11 | GCCG- | | Cy5 | |
| | CATCTAATGAGG | | | |
| | TCGAGGAGTCA | | | |
| 13 | CCGCGCGTCTT- | | Atto 488 | |
| | GGAGCAGTCCTT- | | | |
| | GTT | | | |
| 15 | AACAAGGACTGC | BMN-Q-535 | C3 spacer: | 8 = dT-BMN-Q-535 |
| | TCCAAGAC- | | Chemical block | NHS |
| | GCGCG- | | group comprising | |
| | GAG8GACTCCTC | | a chain of three | |
| | GACCTCATT- | | carbon atoms to | |
| | AGATGCGGCAG- | | prevent unspe- | |
| | CACCTGG- | | cific polymerase | |
| | GACATCGACTATT | | extension of the | |
| | | | strand. | |

Mediators

| | |
|---|---|
| 16 | CACATGCTCATG |
| | TTGTGTGATCG |
| 17 | GTCATCGTGTTC |
| | CTCACATGCTA |
| 18 | ATAGTCGATGTC |
| | CCAGGTGC |

### Two-Part Reporter for the Optimization of Fluorescence Signals

In embodiments, the target sequence-unspecific modular reporter complex can consist of a base strand with a label at the 5′-end and a signal initiation strand with a label at the 3′-end (FIG. 4B). A fluorophore and a quencher are attached to each of the label sites, whereby no fluorescence signal is generated in the initial state. By extending the mediator during the PCR reaction by means of the PCR polymerase, preferably with exonuclease activity, the binding between the base strand and the signal strand (signal oligo) is broken or the binding of the fluorophore is broken so that a signal is generated.

Both the quenching efficiency and the fluorescence intensity of different dyes can thus be optimized for PCR applications, as only one strand needs to be exchanged at a time and can also be used directly (FIG. 4B). The system is ideally suited for this purpose, as the behavior and performance parameters of the modifications and labels can change slightly depending on the sequence and linkage variant. In addition, the target sequence-unspecific modular reporter complex used in this way is considerably less expensive than probe systems of the prior art and performs at least as well in PCR and digital PCR as one-part universal reporters of the prior art. This was confirmed by the results of comparative tests, as shown in FIGS. 6 (PCR analysis) and 7 (dPCR analysis). Accordingly, the combination of fluorophores and quenchers can be optimized much more efficiently by the target sequence-independent modular reporter complex according to the invention in comparison to a one-part double-labeled oligonucleotide (prior art). In the present test, the two-part target sequence-unspecific reporter for the optimization of fluorescence signals shows a very good and improved performance both in comparison to the universal reporter currently used in the prior art and with regard to its use for the investigation of optimal fluorophore-quencher combinations. The results of a first comparison test, which are shown in FIG. 6, demonstrate that the two-part system works surprisingly well despite the non-covalent connection between fluorophore and quencher via several nucleotides of the same molecule. As can be seen in FIG. 6, the curve of the two-part modular reporter with a quencher-labeled base strand and a fluorophore-labeled signal initiation strand (FIG. 6, curve with crosses) and the curve of the universal reporter of the prior art (FIG. 6, curve with triangles) lie exactly on top of each other. The system according to the invention not only enables successful detection of target sequences, but also enables simultaneous detection of a plurality of target sequences. Thus, the result proves that a plurality of fluorophores and quenchers can be used to vary the basic signal and thus, for example, to multiplex monochromatically (e.g. using the same fluorescence color). This is illustrated using the curve of the reporter complex according to the invention with one quencher and two fluorophores (FIG. 6, curve with circles, “multiplex variant 1”) and the curve of the reporter complex according to the invention with two quenchers and two fluorophores (FIG. 6, curve with triangles, “multiplex variant 2”), in each case in double evaluation.

Another surprising result is the optimal functioning of the two-part reporter in digital PCR (dPCR). An example of this is shown in the test result in FIG. 7 and comprises a digital PCR test in the green detection channel by comparing the two-part reporter (FIG. 7, bars “C1”/“C2”) to a universal reporter (UR) of the prior art (FIG. 7, bars “A1”/“A2”). Both the negative amplification control and the positive amplification control show that the system according to the invention was able to successfully detect the same amount of positive droplets as the prior art Universal Reporter (UR).

### Base Strand With Double-Labeled Signal Initiation Strands

The use of a base strand without label also offers advantages. On the one hand, production is simplified and less expensive. On the other hand, various signal initiation molecules (or “signal molecules” for short), which in this case carry a fluorophore and quencher, can also be attached. For example, probe systems such as the TaqMan probe or Molecular Beacons can be connected to a base strand, whereby the signal generation remains target sequence-unspecific, but already incorporates easily available prior art systems. The results of an example test with one embodiment are shown in FIG. 8, the simplified functional diagram of this representative embodiment can be found in FIG. 4A. By binding a plurality of double-labeled signal oligos, for example, much stronger signal changes can be generated per complex than was previously possible with one-part DNA probes or reporter molecules of the prior art. Another surprising result was obtained by using an unlabeled base strand and a signal oligo similar or equivalent to the “TaqMan probe” (FIG. 8, curve with circles). It could be shown that the modular system according to the invention generates a very good signal in this case and can be successfully used for the detection of target sequences. This was also proven in comparison with the prior art universal reporter (UR) in three copies (triplicates) and is shown in FIG. 8 (curve with triangles).

### Multiplex Variations

For direct multiplexing, i.e. the simultaneous detection of different target sequences in a sample and in a reaction, either a plurality of optical channels, further process steps or a complex concentration adjustment or modification of the reporter molecules are necessary according to the prior art. In contrast to this, the reporter according to the invention provides the possibility of combined measurement via different channels, for which purpose base strands according to the invention with more than one receptor complex can be used. In these embodiments, the receptor complexes are preferably offset along the base strand so that they activate different signal complexes (FIG. 3). The possible different color and/or intensity combinations of the respective labels can preferably encode different extended mediator sequences and thus different detected target sequences.

In embodiments, different fluorescence intensities of a plurality of target sequences can be detected simultaneously by using different target sequence-unspecific modular reporter complexes. Here, these target sequence-unspecific modular reporter complexes each have different mediator binding sites on the base strand, each with a different number of fluorescent and/or quencher labels. Various embodiments of this are shown in FIGS. 5A, B and C. In this way, signals of a different signal strength are generated from each signal complex type, which can thus be distinguished in a fluorescence channel (see the results of an example test in FIG. 6). These embodiments offer considerable advantages, particularly in digital PCR, for making different signal clusters distinguishable and represent an improvement on the prior art.

FIG. 9 describes a further embodiment of such a multiplex variant in which, in addition to two reporter complexes (FIGS. 9A and B), each of which has a different mediator binding sequence and a different signal oligo binding site for a different signal oligo with a different fluorophore that generates a signal change when activated by the respective mediator in a different channel, a third reporter complex (FIG. 9C) is added. This is activated by a further different mediator, but also has two binding sites for the respective signal oligos of reporter complexes 1 and 2. In the prior art, corresponding reporter molecules which have two fluorophore and two quencher molecule modifications as well as a 3′ block group are very complex to produce and must also be newly synthesized for each new target sequence. In contrast, the synthesis of a modular reporter according to the invention is less complex and also offers the possibility of freely combining various signal oligos available in the laboratory with a base strand in the test setup without the need for further synthesis. This represents a clear advantage, e.g. with regard to the optimization effort and the costs to be incurred, compared to the probe systems of the prior art. When this reporter complex is activated, a simultaneous signal can be generated in both detection channels. In the case of a digital PCR, where statistically only a single DNA copy is present in a reaction chamber (e.g. droplet), three DNA target sequences can thus be distinguished with only two available color channels in one detection device by cleaving the respective mediator probe in the presence of the respective DNA sequence by means of a mediator probe multiplex PCR according to the invention and thus releasing the corresponding mediator. It is clear to the person skilled in the art how a corresponding digital PCR is to be carried out. Accordingly, the respective change in the fluorescence signals can be used to infer the respective DNA sequence:

TABLE 3
Description of the multiplex approach, which results from
the use of three modular reporter complexes that can bind
two different signal oligos in different proportions.

| DNA target sequence | Signal change | Signal change |
|---|---|---|
| designation/numbering | in channel 1 | in channel 2 |
| Target sequence 1 | Yes | No |
| Target sequence 2 | No | Yes |
| Target sequence 3 | Yes | Yes |
| None | No | No |

Compared to the prior art, using the method described makes it much easier to generate corresponding signal patterns using different combinations of a basic set of base strands and signal oligos.

### FIGURES

The invention is further described by the following figures. These are not intended to limit the scope of the invention, but represent preferred embodiments of aspects of the invention provided to illustrate the invention described herein. p FIG. 1: The figure shows the mediator-probe cleavage during a PCR with activation of the mediator in one embodiment of the invention.

FIG. 2: A) shows signal generation by activation of the signal oligo at a target sequence-unspecific modular universal reporter complex in one embodiment of the invention. B) shows the schematic structure of an embodiment of a target sequence-unspecific modular universal reporter complex according to the invention.

FIG. 3: The figure shows a target sequence-unspecific modular reporter complex with alternating signal and receptor complexes, which enable, for example, mediator signal coding for monochrome multiplexing.

FIG. 4: The figure shows embodiments of different label positions of fluorophore and quencher. A) shows an example of cis-labels on the same signal oligo. B) shows an example of trans-labels.

FIG. 5: The figure shows embodiments for modeling the signal strength (fluorescence signal strength) by multiple labels of a target sequence-unspecific modular reporter complex or by using different quencher strengths. A) shows an example of cis-labels wherein one quencher and one fluorophore are present on a base strand and the signal oligo respectively. B) Shows another example of cis-labels wherein one quencher is present on the base strand and two signal oligos are present with one fluorophore each. C) shows an example of cis-labels, wherein two quenchers and two fluorophores are present on the base strand and signal oligos respectively.

FIG. 6: Comparison of the modular reporter according to the invention with a universal reporter (UR) of the prior art (curve with triangles). The figure shows the optimal functioning of the two-part reporter according to the invention (curve with crosses) in comparison to the UR. The advantageous properties of the reporter according to the invention also offer possibilities for multiplexing detection reactions, e.g. by using reporters according to the invention with labels with different fluorescence strengths, e.g. by means of one quencher and two fluorophores (multiplex variant 1, curve with circles) or by means of two quenchers and two fluorophores (multiplex variant 2, curve with squares). NTCs (non-template control) are shown in dark gray.

FIG. 7: Illustration of the dPCR test results of an embodiment of the two-part reporter according to the invention in comparison to a prior art UR. The two-part reporter according to the invention (NTC: C1, PTC: C2) showed quantitatively the same results as the UR (NTC: A1, PTC: A2) with regard to positive and negative droplets of the dPCR.

FIG. 8: Comparison of the universal reporter (UR) of the prior art (curve with triangles) with the modular reporter according to the invention. An embodiment is shown with an unlabeled base strand and a double-labeled signal oligo (curve with circles), which resembles a “TaqMan probe” (NTCs are shown in dark gray).

FIG. 9: The figure shows a further embodiment of a multiplex variant according to the invention, in which, in addition to two reporter complexes (A and B), each of which has a different mediator binding sequence and a different signal oligo binding site for a different signal oligo with a different fluorophore which generates a signal change when activated by the respective mediator in a different channel, a third reporter complex (C) is added. This is activated by another different mediator, but also has two binding sites for the respective signal oligos of reporter complexes 1 and 2.

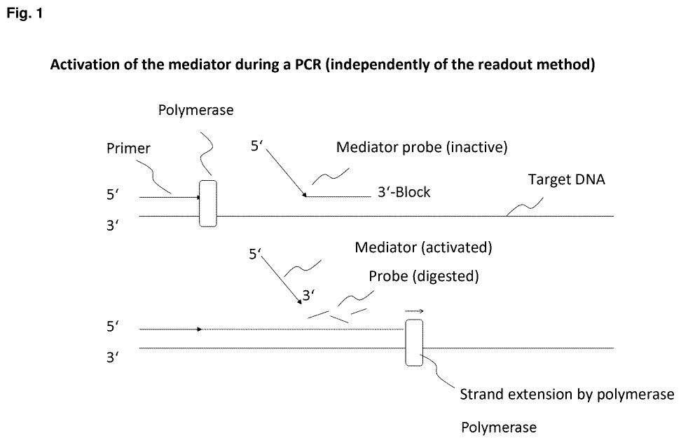

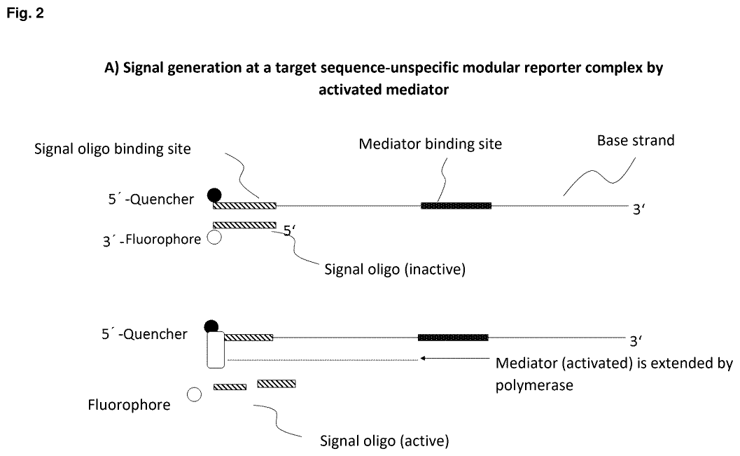

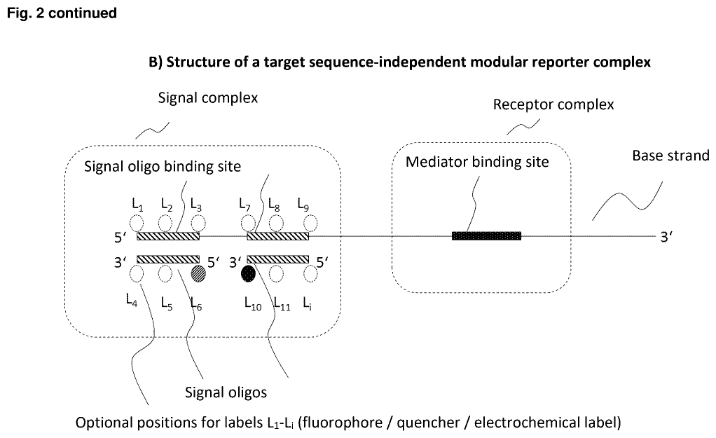

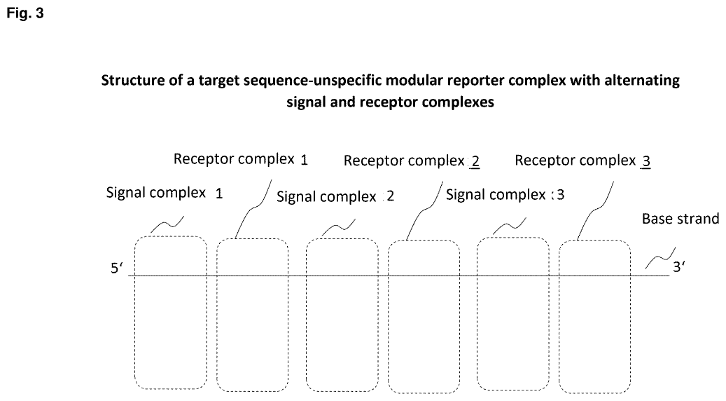

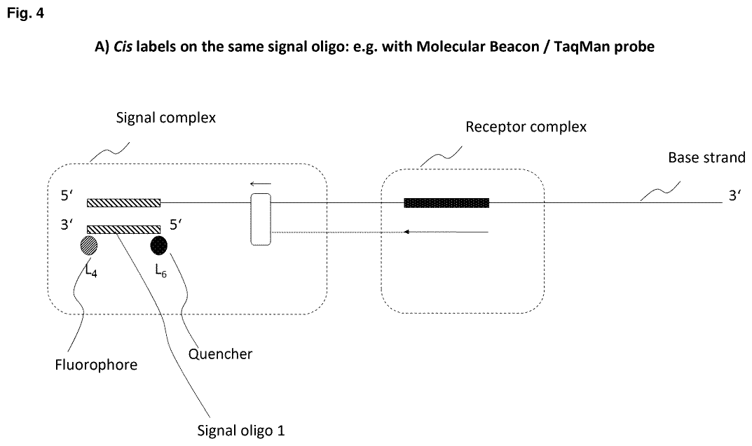

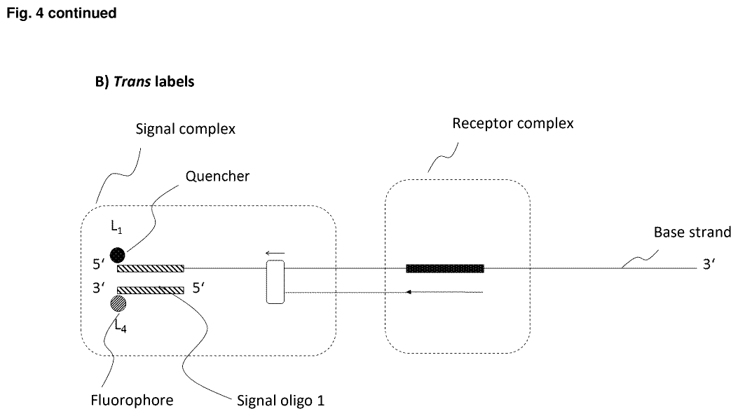

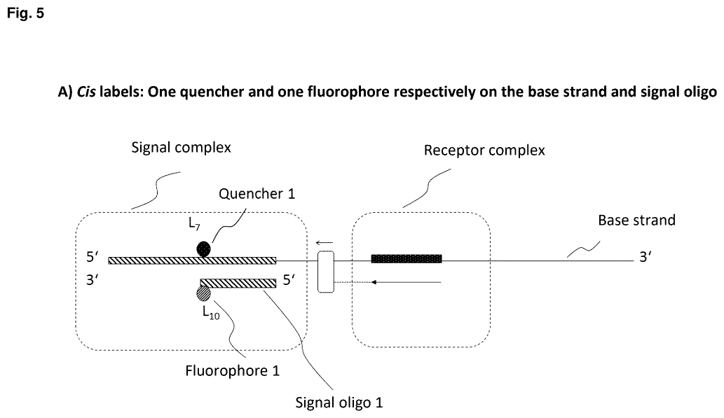

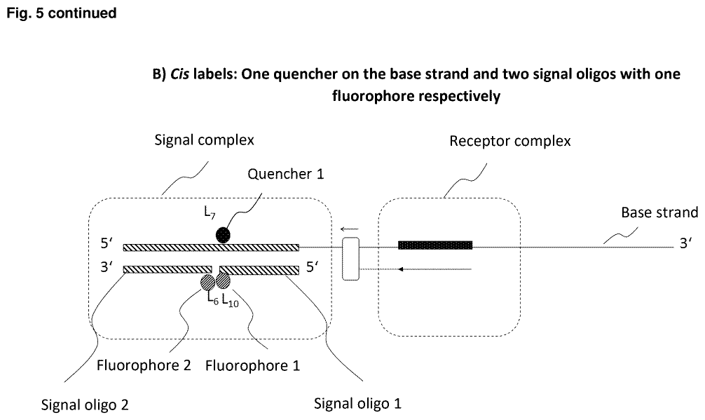

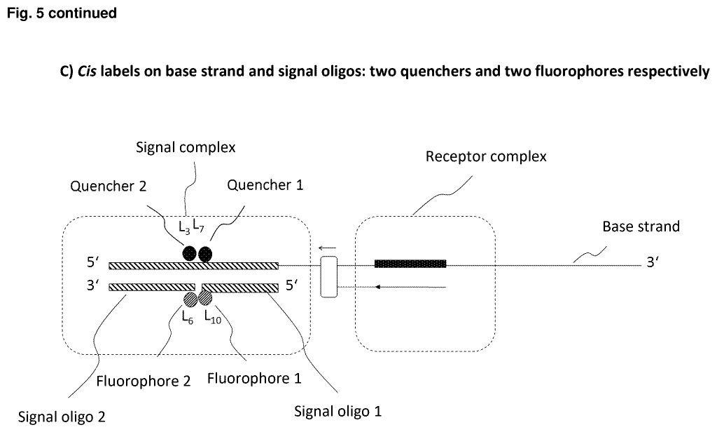

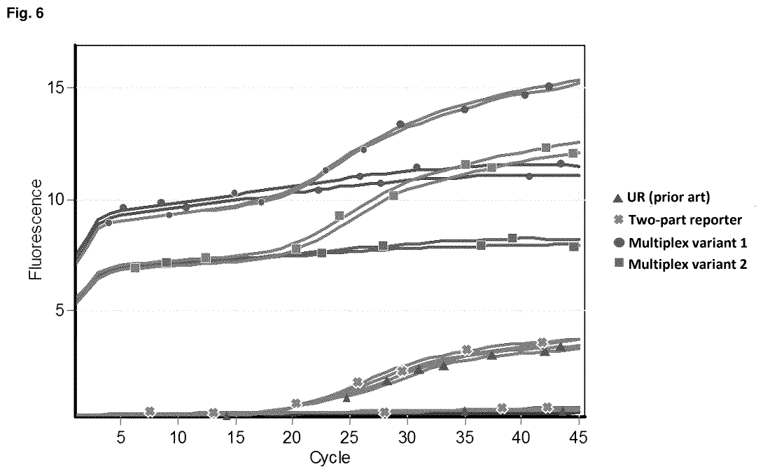

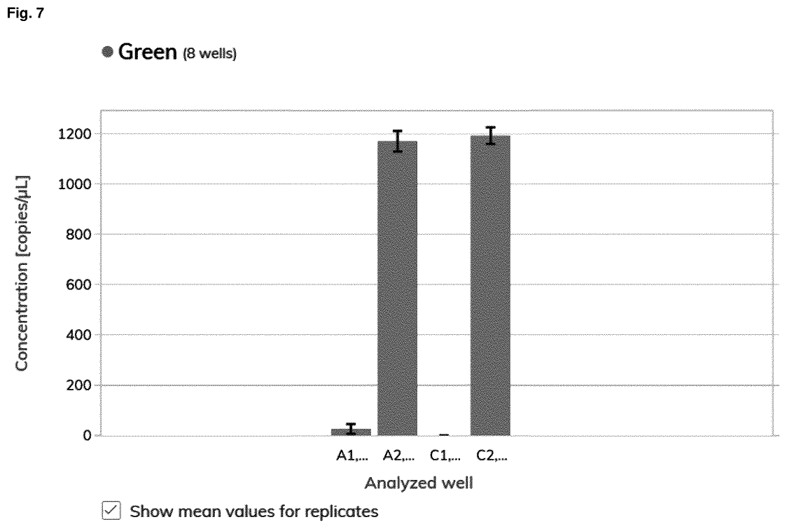

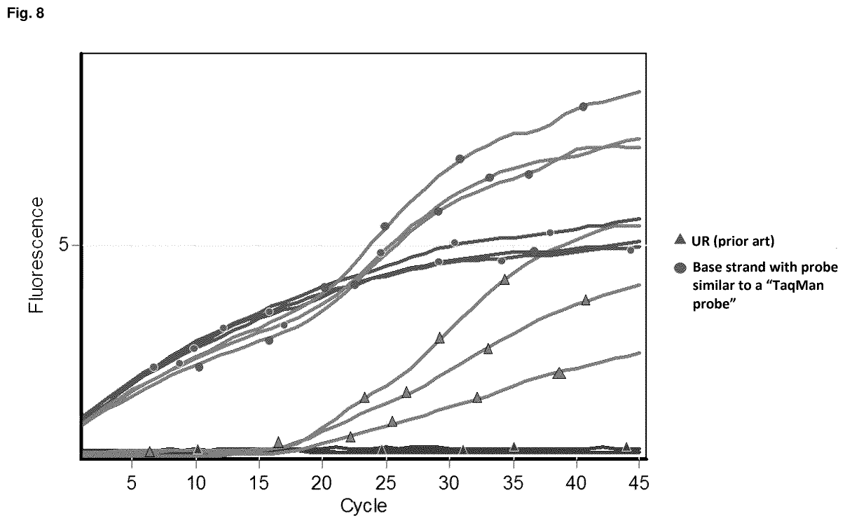

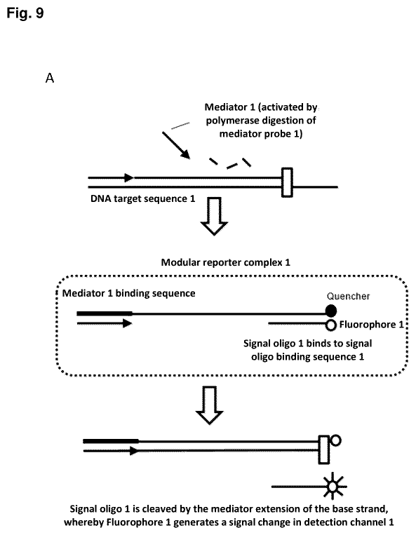

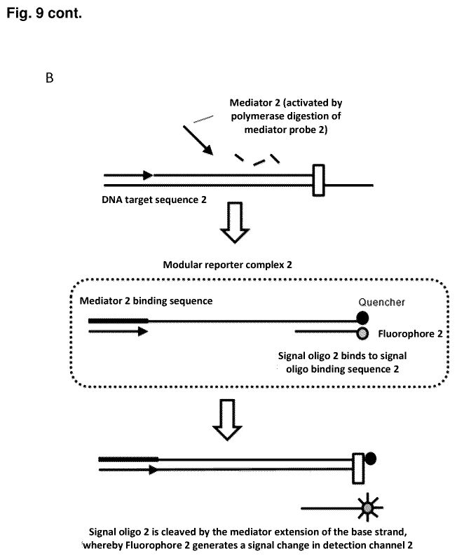

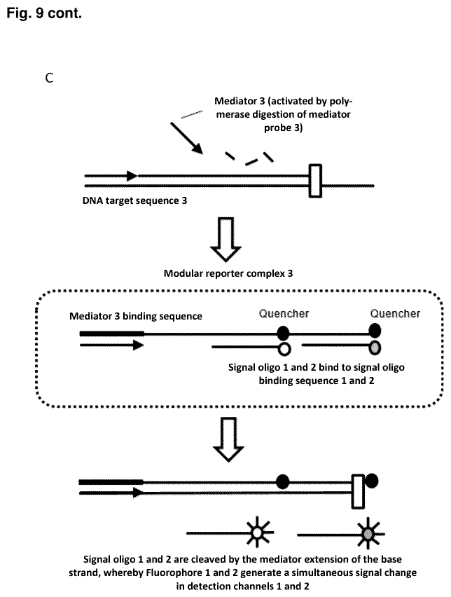

### REFERENCES

Becherer, Lisa; Bakheit, Mohammed; Frischmann, Sieghard; Stinco, Silvina; Borst, Nadine; Zengerle, Roland; Stetten, Felix von (2018): Simplified Real-Time Multiplex Detection of Loop-Mediated Isothermal Amplification Using Novel Mediator Displacement Probes with Universal Reporters. In: Anal. Chem. 90 (7), p. 4741-4748. DOI: 10.1021/acs.analchem.7b05371.
Faltin, Bernd; Wadle, Simon; Roth, Günter; Zengerle, Roland; Stetten, Felix von (2012): Mediator probe PCR: a novel approach for detection of real-time PCR based on label-free primary probes and standardized secondary universal fluorogenic reporters. In: Clinical Chemistry 58 (11), p. 1546-1556. DOI: 10.1373/clinchem.2012.186734.
Faltin, Bernd; Zengerle, Roland; Stetten, Felix von (2013): Current Methods for Fluorescence-Based Universal Sequence-Dependent Detection of Nucleic Acids in Homogenous Assays and Clinical Applications. In: Clinical Chemistry 59 (11), p. 1567-1582. DOI: 10.1373/clinchem.2013.205211.
Heid, C. A.; Stevens, J.; Livak, K. J.; Williams, P. M. (1996): Real time quantitative PCR. In: Genome Research 6 (10), p. 986-994. DOI: 10.1101/gr.6.10.986.
Holland, P. M.; Abramson, R. D.; Watson, R.; Gelfand, D. H. (1991): Detection of specific polymerase chain reaction product by utilizing the 5′- 3′ exonuclease activity of Thermus aquaticus DNA polymerase. In: Proceedings of the National Academy of Sciences of the United States of America 88 (16), p. 7276-7280. DOI: 10.1073/pnas.88.16.7276.
Lehnert, Michael; Kipf, Elena; Schlenker, Franziska; Borst, Nadine; Zengerle, Roland; Stetten, Felix von (2018): Fluorescence signal-to-noise optimisation for real-time PCR using universal reporter oligonucleotides. In: Anal. Methods 10 (28), p. 3444-3454. DOI: 10.1039/C8AY00812D.
Li, Yongsheng; Zhou, Xiaoyan; Ye, Duyun (2008): Molecular beacons: an optimal multifunctional biological probe. In: Biochemical and biophysical research communications 373 (4), p. 457-461. DOI: 10.1016/j.bbrc.2008.05.038.
Liu, Shufeng; Fang, Li; Wang, Yanqun; Wang, Li (2017): Universal Dynamic DNA Assembly-Programmed Surface Hybridization Effect for Single-Step, Reusable, and Amplified Electrochemical Nucleic Acid Biosensing. In: Anal. Chem. 89 (5), p. 3108-3115. DOI: 10.1021/acs.anal-chem.6b04871.
Lyamichev, V.; Brow, M. A.; Dahlberg, J. E. (1993): Structure-specific endonucleolytic cleavage of nucleic acids by eubacterial DNA polymerases. In: Science (New York, N.Y.) 260 (5109), p. 778-783. DOI: 10.1126/science.7683443.
Rodríguez, Miguel A.; García, Teresa; González, Isabel; Hernández, Pablo E.; Martín, Rosario (2005): TaqMan real-time PCR for the detection and quantitation of pork in meat mixtures. In: Meat science 70 (1), p. 113-120. DOI: 10.1016/j.meatsci.2004.12.005.
Schlenker, Franziska, Elena Kipf, Max Deuter, Inga Höffkes, Michael Lehnert, Roland Zengerle, Felix von Stetten, Florian Scherer, Julius Wehrle, Nikolas von Bubnoff, Peter Juelg, Tobias Hutzenlaub, and Nadine Borst. 2021. “Stringent Base Specific and Optimization-Free Multiplex Mediator Probe ddPCR for the Quantification of Point Mutations in Circulating Tumor DNA” Cancers 13, no. 22:5742. https://doi.org/10.3390/cancers13225742
Tan, Weihong; Wang, Kemim; Drake, Timothy J. (2004): Molecular beacons. In: Current opinion in chemical biology 8 (5), p. 547-553. DOI: 10.1016/j.cbpa.2004.08.010.
Tyagi, S.; Kramer, F. R. (1996): Molecular beacons: probes that fluoresce upon hybridization. In: Nat Biotechnol 14 (3), p. 303-308. DOI: 10.1038/nbt0396-303.
Wadle, Simon; Lehnert, Michael; Schuler, Friedrich; Köppel, René; Serr, Annerose; Zengerle, Roland; Stetten, Felix von (2016): Simplified development of multiplex real-time PCR through master mix augmented by universal fluorogenic reporters. In: BioTechniques 61 (3), p. 123-128. DOI: 10.2144/000114443.
Whale, Alexandra S.; Huggett, Jim F.; Tzonev, Svilen (2016): Fundamentals of multiplexing with digital PCR. In: Biomolecular Detection and Quantification 10, p. 15-23. DOI: 10.1016/j.bdq.2016.05.002.

1. A method for detecting at least one target nucleic acid sequence, comprising:
a) providing at least one target sequence-unspecific modular reporter complex comprising:
at least one label, and
at least two oligonucleotides, comprising
(i) a base strand, comprising
1) at least one mediator binding site,
2) at least one signal oligo binding site, and
(ii) at least one signal oligo,
wherein the at least one signal oligo binding site of the base strand and the at least one signal oligo hybridize with each other but are not covalently linked and together form a signal complex,
b) providing at least one mediator probe, wherein the mediator probe comprises an oligonucleotide having at least one probe sequence and at least one mediator sequence, wherein the at least one probe sequence exhibits an affinity for the at least one target nucleic acid sequence, and the at least one mediator sequence exhibits an affinity for the at least one mediator binding site on the base strand of the at least one target sequence-unspecific modular reporter complex,
c) performing a PCR amplification of the at least one target nucleic acid sequence,
d) binding of the at least one a probe sequence of the at least one mediator probe to the at least one target nucleic acid sequence,
e) cleaving the at least one probe sequence of the at least one mediator probe bound to the at least one target nucleic acid sequence via a PCR polymerase with nuclease activity during the PCR amplification, wherein the mediator sequence is released to produce a released mediator sequence,
f) binding the at least one released mediator sequence to the at least one mediator binding site of the at least one target sequence-unspecific modular reporter complex,
g) extending the sequence of at least one mediator sequence bound to the at least one mediator binding site via a PCR polymerase, wherein
a bond of the hybridized at least one signal oligo binding site and at least one signal oligo is broken, thereby initiating a signal change,
h) detection of at least one signal change as evidence of a presence of the at least one target nucleic acid sequence.

2. The method according to claim 1, wherein the at least one label comprises at least one fluorophore and/or at least one quencher.

3. The method according to claim 2, wherein no fluorescence signal change is generated by the at least one fluorophore when the at least one signal oligo is hybridized to the at least one signal oligo binding site of the base strand,
wherein either the at least one quencher is localized at the at least one signal oligo binding site of the base strand and the at least one fluorophore is localized at the at least one signal oligo or vice versa, and
wherein in g) the at least one fluorophore and the at least one quencher are separated, thereby initiating the signal change.

4. The method according to claim 2, wherein the at least one label comprises the at least one fluorophore and the at least one quencher, and
wherein both, the at least one quencher and the at least one fluorophore, are localized on the at least one signal oligo, and
wherein in g) the at least one signal oligo is cleaved by the PCR polymerase, whereby the at least one fluorophore and the at least one quencher are separated, thereby initiating the at least one signal change.

5. The method according to claim 1, wherein the base strand comprises the at least one signal oligo binding site to which two or more of the signal oligos are hybridized, and wherein the two or more signal oligos and/or the base strand have one or more labels at the at least one signal oligo binding site.

6. The method according to claim 1, wherein the base strand comprises the two or more signal oligo binding sites, and wherein at least one of the signal oligos hybridized to the two or more signal oligo binding sites and/or
the base strand has one or more of the at least one label at at least one of the two or more signal oligo binding sites.

7. The method according to claim 1, wherein the base strand comprises two or more of the signal oligo binding sites which together form a signal complex and to each of which at least one of the at least one signal oligo is hybridized with at least one label is hybridized, wherein the signal change generated by the labels of the signal complex is characteristic of the at least one nucleic acid target sequence.

8. The method according to claim 1, wherein the base strand comprises at least two or more of the signal complexes each having different signal oligos and/or different labels on the signal oligos, and wherein the base strand comprises the at least one mediator binding site corresponding to the at least two or more signal complexes.

9. The method according to claim 1, wherein the at least one target nucleic acid sequence comprises a first and a second target nucleic acid sequence, the at least one mediator probe comprises a first and second mediator probe, wherein the at least one target sequence-unspecific modular reporter complex enables the detection of at least the first and the second target nucleic acid sequence, wherein the at least one mediator binding site of the base strand,
comprises at least a first and a second mediator binding site for at least a first and a second mediator sequence of at least the first and the second mediator probe, and at least a first and a second signal oligo binding site to which at least a first and a second signal oligo is hybridized, and
wherein the first mediator probe comprises a probe sequence having an affinity for the first target nucleic acid sequence and the second mediator probe comprises a probe sequence having an affinity for the second target nucleic acid sequence.

10. The method according to claim 9, wherein the base strand comprises at least a first and a second label, wherein the signal change due to the at least one first label is characteristic of the first target nucleic acid sequence, and the signal change due to the at least one second label is characteristic of the second target nucleic acid sequence.

11. The method according to claim 1, wherein the at least one target nucleic acid sequence comprises a first and second target nucleic acid sequence in a) at least a first and a second target sequence-unspecific modular reporter complex is provided,
wherein the at least first target sequence-unspecific modular reporter complex enables the detection of at least the first target nucleic acid sequence and the at least second target sequence-unspecific modular reporter complex enables the detection of the second target nucleic acid sequence, wherein the signal change due to the at least one label of the at least first target sequence-unspecific modular reporter complex is characteristic of the first target nucleic acid sequence and the signal change due to the at least one label of the at least second target sequence-unspecific modular reporter complex is characteristic of the second target nucleic acid sequence.

12. The method according to claim 11, wherein the signal changes characteristic of the at least first and the at least second target nucleic acid sequences differ from each other by their color and/or their fluorescence or signal strength.

13. The method according to claim 1, wherein
c) to h) are carried out as part of a reaction selected from the group consisting of PCR, digital PCR, RT-PCR, digital RT-PCR, real-time/qPCR, droplet PCR, or and any combination thereof.

14. The method according to claim 1, wherein the detection of the signal change comprises analyzing the signal change as a function of the a detection temperature.

15. A kit comprising:
at least one oligonucleotide primer
at least one mediator probe
at least one signal oligo
at least one base strand
at least one buffer
PCR polymerase, wherein the kit is adapted to carry out the method according to claim 1.
